(19) United States
(12) Patent Application Publication
(10) Pub. No.: US 2003/0203408 A1
(43) Pub. Date: Oct. 30, 2003

(54) Computer-implemented method for identifying peaks in electropherogram data

(76) Inventors: Stephen Williams, San Carlos, CA (US); Paul E. Theobald, Pleasanton, CA (US); Mengxiana Tang, San Jose, CA (US)

Correspondence Address: ACLARA BIOSCIENCES, INC., 1288 PEAR AVENUE, MOUNTAIN VIEW, CA, 94043, US

(21) Appl. No.: 10/405,227

(22) Filed: Apr. 1, 2003

Related U.S. Application Data
(60) Provisional application No. 60/369,652, filed on Apr. 2, 2002.

(51) Int. Cl. G01N 33/53; G06F 19/00; G01N 33/48; G01N 33/50

(52) U.S. Cl. 435/7.1; 702/19

(57) ABSTRACT

The invention provides methods and algorithms for measuring one or more analytes in a sample by using a plurality of releasable molecular tags attached to binding compounds specific for the analytes of interest. After binding compounds specifically bind to their respective analytes to form complexes, the molecular tags of the binding compounds forming such complexes are cleaved and released, while the molecular tags of those binding compounds not forming such complexes are not released. The released molecular tags are then electrophorectically separated along with one or more electrophorectic standards to generate electropherogram data, and the identity of each molecular tag is determined by the location of its corresponding peak in such data relative to the one or more of the electrophorectic standards. In this way, distortions in the electropherogram data due to factors such as instrumentation differences, assay conditions, reagent variability, or the like, can be taken into account and experimental results from different electrophoresis systems, different assays, or the like may be compared.

### CROSS-REFERENCE TO RELATED APPLICATIONS AND PATENTS

This application claims priority from U.S. provisional application Ser No. 60/369,652 filed 2 Apr. 2002, which is incorporated herein by reference in its entirety.

### FIELD OF THE INVENTION

This invention relates to method for detecting and/or measuring one or more analytes in an assay by the generation and electrophorectic separation of molecular tags.

### BACKGROUND OF THE INVENTION

The development of several powerful technologies for genome-wide and proteome-wide expression measurements has created an opportunity to study and understand the coordinated activites of large sets of, if not all, an organism's genes in response to a wide variety of conditions and stimuli, e.g. DeRisi et al, Science, 278: 680-686 (1997); Wodicka et al, Nature Biotechnology, 15: 1359-1367 (1997); Velculescu et al, Cell, 243-251 (1997); Brenner et al, Nature Biotechnology, 18: 630-634 (2000); Mcdonald et al, Disease Markers, 18: 99-105 (2002); Patterson, Bioinformatics, 18 (Suppl 2): S181 (2002). Studies using these technologies have shown that subsets of genes appear to be co-regulated to perform particular functions and that subsets of expressed genes and proteins can be used to classify cells phenotypically, e.g. Shiffman and Porter, Current Opinion in Biotechnology, 11; 598-601 (2000); Afshari et al, Nature, 403: 503-511 (2000); Golub et al, Science, 286: 531-537 (1999); van't Veer et al, Nature, 415: 530-536 (2002); and the like. This has led to an interest in measuring the messenger RNA or protein expression of subsets of genes that number in the range of from 3-4 to several tens, or more.

For example, an area of interest in drug development is the expression profiles of genes and proteins involved with the metabolism or toxic effects of xenobiotic compounds. Several studies have shown that sets of several tens of genes can serve as indicators of compound toxicity, e.g. Thomas et at, Molecular Pharmacology, 60: 1189-1194 (2001); Waring et al, Toxicology Letters, 120: 359-368 (2001); Longueville et al, Biochem. Pharmacology, 64:137-149 (2002); and the like. Similarly, in the area of cancer diagnostics and prognosis, the differential expression of small sets of genes or proteins has been shown frequently to have strong correlations with the progression and prognosis of a cancer, e.g. Bunn et al, Semin. Oncol., 29 (5 Suppl 14): 38-44 (2002); Baker, Oncogene, 17: 3261-3270 (1998); Yarden, Oncology, 61: Suppl. 2:1-13 (2001); Ouyang et al, Lancet, 353:1591-1592 (1999); George et al, Nature Reviews Drug Discovery, 1: 808-820 (2002); Howard et al, Trends in Pharmaceutical Sciences, 22: 132-140 (2001); Seymour, Current Drug Targets, 2: 117-133 (2001).

Recently, Singh and co-workers have developed a technology for medium-scale multiplexed assays well suited for the above measurements. The technology utilizes libraries of releasable molecular tags, differentiated by electrophoretic mobility and optical characteristics, that are attached to binding agents for multiplexed detection or quantification of analytes, e.g. International patent publications WO 00/66607; WO 01/83502; WO 02/95356; WO 03/06947; and U.S. Pat. Nos. 6,322,980 and 6,514,700. Implementation of the technology has posed challenges, however, because even though the separation properties of molecular tags are selected beforehand, particular assays and instrumentation, as well as the separation process itself, introduce to difficult-to-predict distortions into the separation profile of the molecular tags, including peak and trace stretching, peak shifts, baseline changes, and the like. Analysis of such data is further complicated because molecular tags may or may not be present in the separation mixture depending on whether or not their associated analytes are present in a sample being assayed.

In view of the above, the availability of a convenient and cost effective technique for measuring the presence or absence or quantities of multiple analytes, such as protein of mRNA gene expression products, in a single assay reaction would advance many fields where such measurements are becoming increasingly important, including life science research, medical research and diagnostics, drug discovery, genetic identification, animal and plant science, and the like.

### SUMMARY OF THE INVENTION

The present invention is directed to methods and algorithms for analyzing an electropherogram of a plurality of molecular tags whose electrophoretic separation and measurement provide information about the presence or quantity of analytes in a sample. In one aspect of the invention, such analysis is carried out with the following steps: (a) reading electropherogram data from a storage medium, the electropherogram data obtained by electrophoretic separation of the plurality of molecular tags and one or more electrophoretic standards, each electrophoretic standard and molecular tag having a different electrophoretic mobility such that upon electrophoretic separation each electrophoretic standard and molecular tag forms a distinct peak in the electropherogram data, and each peak of such one or more electrophoretic standards and molecular tags having a migration interval and each migration interval having a mean; (b) determining a peak location of at least one electrophoretic standard within a migration interval in the electropherogram data; (c) determining peak locations of peaks within a migration interval in the electropherogram data closest to an electrophoretic standard or a qualified peak in a qualified peak set, the peak locations being relative to the location of the closest electrophoretic standard or qualified peak, and correlating with a molecular tag or an electrophoretic standard a peak within the migration interval whose location is closest to the mean of the migration interval; (d) determining a peak signal-to-noise ratio of the peak and adding the peak to the qualified peak set if the peak signal-to-noise ratio is greater than or equal to 1.5; and (e) repeating steps (c) and (d) until a peak location is correlated to every molecular tag having a peak in the electropherogram data.

In one aspect, the invention provides computer-readable products and computer systems for implementing the steps of the above method.

The present invention provides a method of detecting or measuring a plurality of analytes that has several advantages over current techniques including, but not limited to, (1) the detection and/or measurement of molecular tags that are separated from an assay mixture provide greatly reduced background and a significant gain in sensitivity; (2) the use of molecular tags that are specially designed for ease of separation and detection thereby providing convenient multiplexing capability; and (3) the accurate detection and quantification of peaks in electropherogram data correlated to molecular tags by using the molecular tags themselves as standards.

### BRIEF DESCRIPTION OF THE DRAWINGS

FIG. 1A illustrates an exemplary multiplexed assay for detecting or measuring target analytes, such as proteins, by generating molecular tags in a “sandwich” type of assay using antibodies as binding compounds.

FIG. 1B illustrates an exemplary multiplexed assay for detecting or measuring target polynucleotides by generating molecular tags in a “taqman” type of assay in a polymerase chain reaction (PCR).

FIG. 1C illustrates an exemplary multiplexed assay for detecting or measuring target polynucleotides by generating molecular tags in an Invader type of assay.

FIGS. 2A through 2K illustrate features of algorithms of the invention.

FIG. 3 is a flow chart illustrating the main steps of a preferred algorithm for identifying peaks in electropherogram data.

FIG. 4A diagrammatically illustrates a method of determining peak parameters of peaks that extend outside the dynamic range in one of the data collection channels of a detection system.

FIG. 4B is a flow chart illustrating the main steps for determining peak parameters of peaks that extend outside the dynamic range in one of the data collection channels of a detection system.

FIG. 5 illustrates the chemical formulas, electrophoretic migration times, and molecular weights of several molecular tags from an assay for detecting polynucleotide analytes.

FIG. 6 illustrates further chemical formulas, electrophoretic migration times, and charges of several molecular tags from an assay for detecting polynucleotide analytes. The moieties in the formulas represented as “C₃,”“C₆,” and “C₉” are 3-, 6-, and 9-carbon polyethylene glycol linkers conjugated to a phosphodiester. For example, C₃ is -((CH₂)₂O)₃-OP(O³¹)(=O)O−.

FIG. 7 illustrates the formulas of ten molecular tags.

FIGS. 8A-8J illustrates formulas of NHS-esters or biotinylated forms of molecular tags that may be conjugated to binding compounds either having a free amine or a biotin.

FIGS. 9A-F illustrate oxidation-labile linkages and their respective cleavage reactions mediated by singlet oxygen.

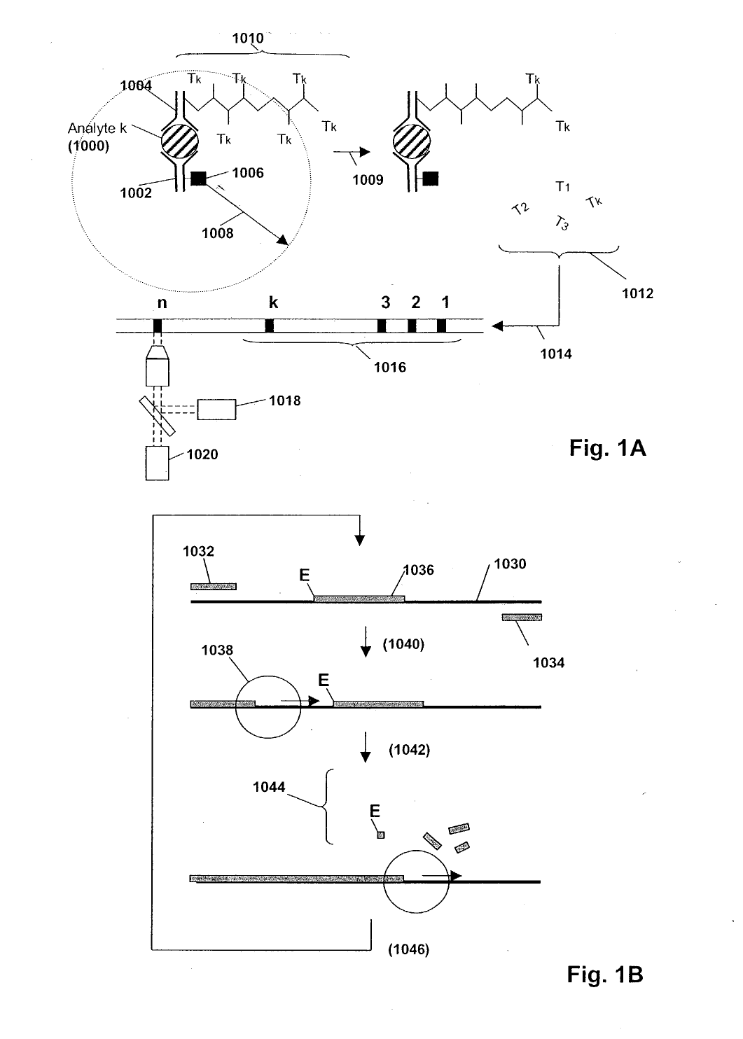

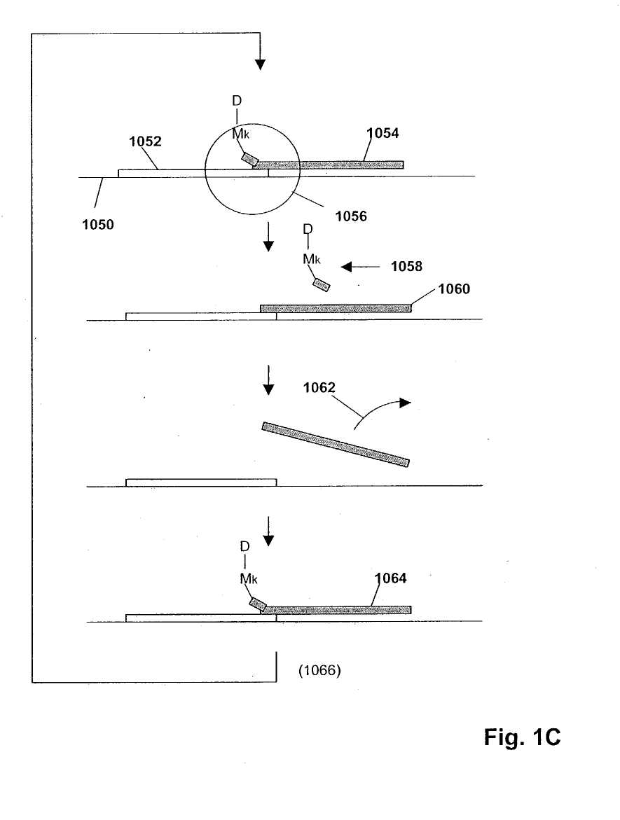

FIGS. 1OA-9C illustrate steps in practicing the method of the invention using a microfluidics capillary electrophoresis (CE) device.

FIG. 11 is an electropherogram showing peaks identified according to molecular tag and associated analyte.

### Definitions

“Analyte” means a substance, compound, or component in a sample whose presence or absence is to be detected or whose quantity is to be measured. Analytes include but are not limited to peptides, proteins, polynucleotides, polypeptides, oligonucleotides, organic molecules, haptens, epitopes, parts of biological cells, posttranslational modifications of proteins, receptors, complex sugars, vitamins, hormones, and the like. There may be more than one analyte associated with a single molecular entity, e.g. different phosphorylation sites on the same protein.

“Antibody” means an immunoglobulin that specifically binds to, and is thereby defined as complementary with, a particular spatial and polar organization of another molecule. The antibody can be monoclonal or polyclonal and can be prepared by techniques that are well known in the art such as immunization of a host and collection of sera (polyclonal) or by preparing continuous hybrid cell lines and collecting the secreted protein (monoclonal), or by cloning and expressing nucleotide sequences or mutagenized versions thereof coding at least for the amino acid sequences required for specific binding of natural antibodies. Antibodies may include a complete immunoglobulin or fragment thereof, which immunoglobulins include the various classes and isotypes, such as IgA, IgD, IgE, IgG1, IgG2a, IgG2b and IgG3, IgM, etc. Fragments thereof may include Fab, Fv and F(ab′)2, Fab′, and the like. In addition, aggregates, polymers, and conjugates of immunoglobunins or their fragments can be used where appropriate so long as binding affinity for a particular polypeptide is maintained.

“Antibody binding composition” means a molecule or a complex of molecules that comprise one or more antibodies and derives its binding specificity from an antibody. Antibody binding compositions include, but are not limited to, antibody pairs in which a first antibody binds specifically to a target molecule and a second antibody binds specifically to a constant region of the first antibody; a biotinylated antibody that binds specifically to a target molecule and streptavidin derivatized with moieties such as molecular tags or photosensitizers; antibodies specific for a target molecule and conjugated to a polymer, such as dextran, which, in turn, is derivatized with moieties such as molecular tags or photosensitizers; antibodies specific for a target molecule and conjugated to a bead, or microbead, or other solid phase support, which, in turn, is derivatized with moieties such as molecular tags or photosensitizers, or polymers containing the latter.

“Binding compound” means any molecule to which molecular tags can be directly or indirectly attached that is capable of specifically binding to a membrane-associated analyte. Binding compounds include, but are not limited to, antibodies, antibody binding compositions, peptides, proteins, particularly secreted proteins and orphan secreted proteins, nucleic acids, and organic molecules having a molecular weight of up to 1000 daltons and consisting of atoms selected from the group consisting of hydrogen, carbon, oxygen, nitrogen, sulfur, and phosphorus.

“Capillary-sized” in reference to a separation column means a capillary tube or channel in a plate or microfluidics device, where the diameter or largest dimension of the separation column is between about 25-500 microns, allowing efficient heat dissipation throughout the separation medium, with consequently low thermal convection within the medium.

“Computer-readable product” means any tangible medium for storing information that can be read by or transmitted into a computer. Computer-readable products include, but are not limited to, magnetic diskettes, magnetic tapes, optical disks, CD-ROMs, punched tape or cards, read-only memory devices, direct access storage devices, gate arrays, electrostatic memory, and any other like medium.

“Electropherogram” in reference to the separation of molecular tags means a chart, graph, curve, bar graph, or other representation of signal intensity data versus a parameter related to the molecular tags, such as migration time, that provides a readout, or measure, of the number of molecular tags of each type produced in an assay. A “peak” or a “band” or a “zone” in reference to an electropherogram means a region where signal intensity values are high, e.g. relative to background, and correspond to a local concentration of a separated compound. There may be multiple separation profiles for a single assay, for example, if molecular tags are labeled with fluorescent dyes and data is collected and recorded at multiple wavelengths. Thus, molecular tags or electrophoretic standards that have nearly identical electrophoretic mobilities may have distinct peaks in electropherogram data because they are labeled with different dyes. In one aspect, released molecular tags are separated by differences in electrophoretic mobility to form an electropherogram wherein different molecular tags correspond to distinct peaks on the electropherogram. A measure of the distinctness, or lack of overlap, of adjacent peaks in an electropherogram is “electrophoretic resolution,” which may be taken as the distance between adjacent peak maximums divided by four times the larger of the two standard deviations of the peaks. Preferably, adjacent peaks have a resolution of at least 1.0, and more preferably, at least 1.5, and most preferably, at least 2.0. In a given separation and detection system, the desired resolution may be obtained by selecting a plurality of molecular tags whose members have electrophoretic mobilities that differ by at least a peak-resolving amount, such quantity depending on several factors well known to those of ordinary skill, including signal detection system, nature of the fluorescent moieties, the diffusion coefficients of the tags, the presence or absence of sieving matrices, nature of the electrophoretic apparatus, e.g. presence or absence of channels, length of separation channels, and the like. As used herein, “electropherogram data” means a table, or discrete function, F(Xᵢ) of signal intensity values for each migration time, Xᵢ, collected in the eletrophorectic separation of molecular tags. Preferably, electroplerogram data comprises fluorescence intensity values collected by conventional detection systems in a capillary electrophoresis instrument.

As used herein, the term “kit” refers to any delivery system for delivering materials. In the context of reaction assays, such delivery systems include systems that allow for the storage, transport, or delivery of reaction reagents (e.g., probes, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. Such contents may be delivered to the intended recipient together or separately. For example, a first container may contain an enzyme for use in assay, while a second container contains probes.

“Polypeptide” refers to a class of compounds composed of amino acid residues chemically bonded together by amide linkages with elimination of water between the carboxy group of one amino acid and amino group of another amino acid. A polypeptide is a polymer of amino acid residues, which may contain a large number of such residues. Peptides are similar to polypeptides, except that, generally, they are comprised of a lesser number of amino acids. Peptides are sometimes referred to as oligopeptides. There is no clear-cut distinction between polypeptides and peptides. For convenience, in this disclosure and claims, the term “polypeptide” will be used to refer generally to peptides and polypeptides. The amino acid residues may be natural or synthetic. “Protein” refers to a polypeptide, usually synthesized by a biological cell, folded into a defined three-dimensional structure. Proteins are generally from about 5,000 to about 5,000,000 or more in molecular weight, more usually from about 5,000 to about 1,000,000 molecular weight, and may include posttranslational modifications, such acetylation, acylation, ADP-ribosylation amidation, covalent attachment of flavin, covalent attachment of heme moiety, covalent attachment of a nucleotide or nucleotide derivative, covalent attachment of a lipid or lipid derivative, covalent attachment of phosphotidylinositol, cross-linking, cyclizaton, disulfide bond formation, demethylation, formation of covalent cross-links, formation of cystine, formation, of pyroglutamate, formylation, gamma-carboxylation, glycosylation, GPI anchor formation, hydroxylation, iodination, methylation, myristoylation, oxidation, phosphorylation, prenylation, racemization, selenoylation, sulfation, and ubiquitination, e.g. World. F., Post-translational Protein Modifications: Perspectives and Prospects, pgs. 1-12 in Post-translational Covalent Modifications of Proteins, B. C. Johnson, Ed., Academic Press, New York, 1983. Proteins include, by way of illustration and limitation, cytokines or interleukins, enzymes such as e.g., kinases, proteases, galactosidases and so forth, protamines, histones, albumins, immunoglobulins, scleroproteins, phosphoproteins, mucoproteins, chromoproteins, lipoproteins, nucleoproteins, glycoproteins, T-cell receptors, proteoglycan, unclassified proteins, e.g., somatotropin, prolactin, insulin, pepsin, proteins found in human plasma, blood clotting factors, blood typing factors, protein hormnones, cancer antigens, tissue specific antigens, peptide hormones, nutritional markers, tissue specific antigens, and synthetic peptides.

The term “sample” in the present specification and claims is used in a broad sense. On the one hand it is meant to include a specimen or culture (e.g., microbiological cultures). On the other hand, it is meant to include both biological and environmental samples. A sample may include a specimen of synthetic origin. Biological samples may be animal, including human, fluid, solid (e.g., stool) or tissue, as well as liquid and solid food and feed products and ingredients such as dairy items, vegetables, meat and meat by-products, and waste. Biological samples may include materials taken from a patient including, but not limited to cultures, blood, saliva, cerebral spinal fluid, pleural fluid, milk, lymph, sputum, semen, needle aspirates, and the like. Biological samples may be obtained from all of the various families of domestic animals, as well as feral or wild animals, including, but not limited to, such animals as ungulates, bear, fish, rodents, etc. Environmental samples include environmental material such as surface matter, soil, water and industrial samples, as well as samples obtained from food and dairy processing instruments, apparatus, equipment, utensils, disposable and non-disposable items. These examples are not to be construed as limiting the sample types applicable to the present invention.

A “sieving matrix” or “sieving medium” means an electrophoresis medium that contains crosslinked or non-crosslinked polymers, which are effective to retard electrophoretic migration of charged species through the matrix.

“Specific” or “specificity” in reference to the binding of one molecule to another molecule, such as a binding compound, or probe, for a target analyte, means the recognition, contact, and formation of a stable complex between the probe and target, together with substantially less recognition, contact, or complex formation of the probe with other molecules. In one aspect, “specific” in reference to the binding of a first molecule to a second molecule means that to the extent the first molecule recognizes and forms a complex with another molecules in a reaction or sample, it forms the largest number of the complexes with the second molecule. In one aspect, this largest number is at least fifty percent of all such complexes form by the first molecule. Generally, molecules involved in a specific binding event have areas on their surfaces or in cavities giving rise to specific recognition between the molecules binding to each other. Examples of specific binding include antibody-antigen interactions, enzyme-substrate interactions, formation of duplexes or triplexes among polynucleotides and/or oligonucleotides, receptor-ligand interactions, and the like. As used herein, “contact” in reference to specificity or specific binding means two molecules are close enough that weak noncovalent chemical interactions, such as Van der Waal forces, hydrogen bonding, ionic and hydrophobic interactions, and the like, dominate the interaction of the molecules. As used herein, “stable complex” in reference to two or more molecules means that such molecules form noncovalently linked aggregates, e.g. by specific binding, that under assay conditions are thermodynamically more favorable than a non-aggregated state.

As used herein, the term “spectrally resolvable” in reference to a plurality of flourescent labels means that the flourescent emission bands of the labels are sufficiently distict, i.e. sufficiently non-overlapping, that molecular tags to which the respective labels are attached can be distinguished on the basis of the flourescent signal generated by the respective labels by standatd photodetection systems, e.g. employing a system of band pass filters and photomultiplier tubes, or the like, as exemplified by the systems described in U.S. Pat. Nos. 4,230,558;4,811,218, or the like, or in Wheeless et al, pgs. 21-76, in Flow Cytometry: Instrumentation and Data Analysis (Academic Press, New York, 1985).

### DETAILED DESCRIPTION OF THE INVENTION

The invention provides methods and algorithms for measuring one or more analytes in a sample by using a plurality of releasable molecular tags attached to binding compounds specific for the analytes of interest. After binding compounds specifically bind to their respective analytes to form complexes, the molecular tags of the binding compounds forming such complexes are cleaved and released, while the molecular tags of those binding compounds not forming such complexes are not released. The released molecular tags are then electrophorectically separated along with one or more electrophoretic standards to generate electropherogram data, and the identity of each molecular tag is determined by the location of its corresponding peak in such data relative to the one or more of the electrophorectic standards. In this way, distortions in the electropherogram data due to factors such as instrumentation difference, assay conditions, reagent variability, or the like, can be taken into account and experimental results from different electrophoresis systems, different assays, or the like, may be compared.

In one aspect, the invention provides computer-implemented methods for correlating peaks detected in electropherogram data with molecular tags used in an assay. In one aspect, the mmethods are implemented by first identifying an electrophorectic standard, then successively identyfying peaks correlated to molecular tags and/or further standards. In another aspect, every molecular tag employed in an assay is also employed as its own standard in a separation mixture. That is, known quantities of each molecular tag are added prior to separation so that for every tag, whether released or not in the assay, there is a peak of known location in the electropherogram data. In this embodiment the presence or quantity of an analyte is determined by subtracting the contribution of the “self-standard” from the observed signal.

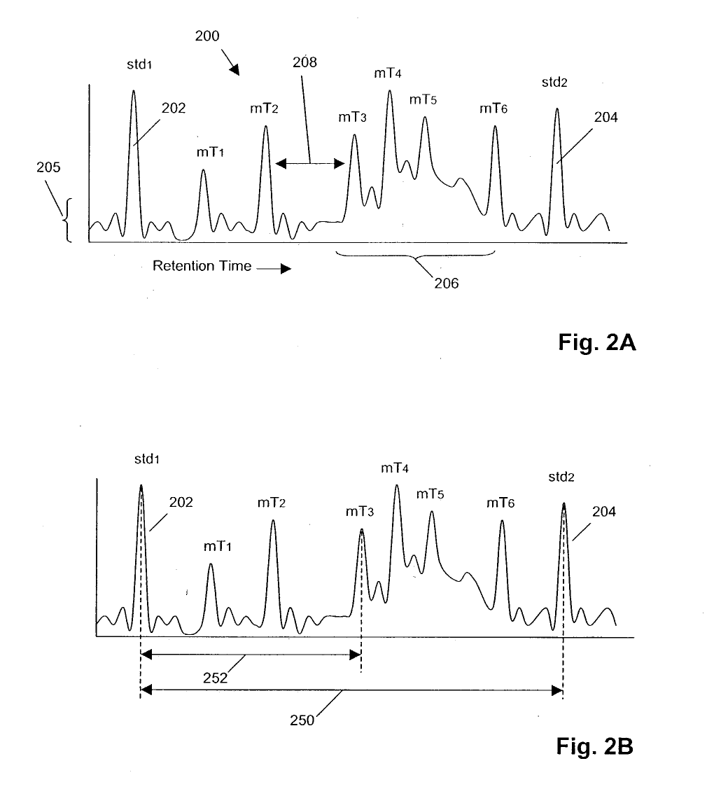

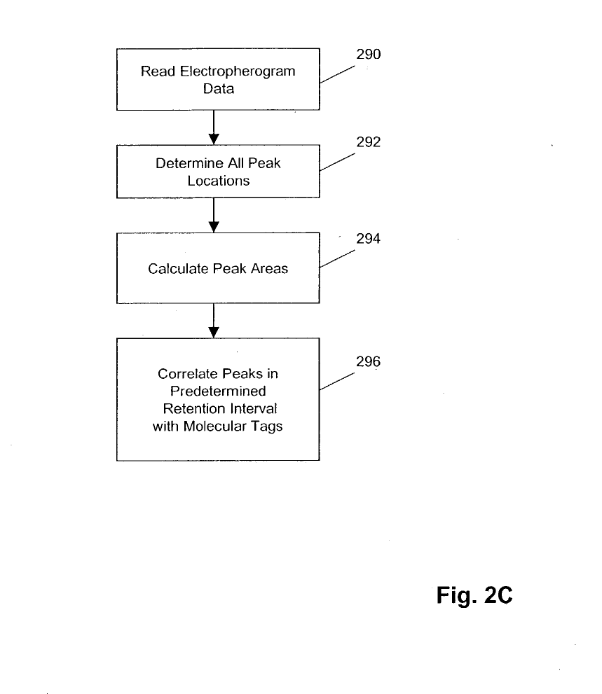

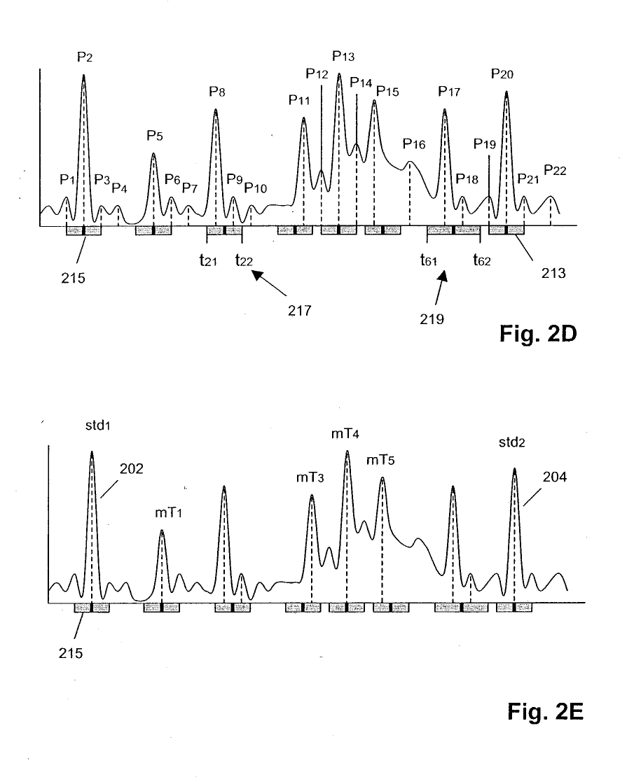

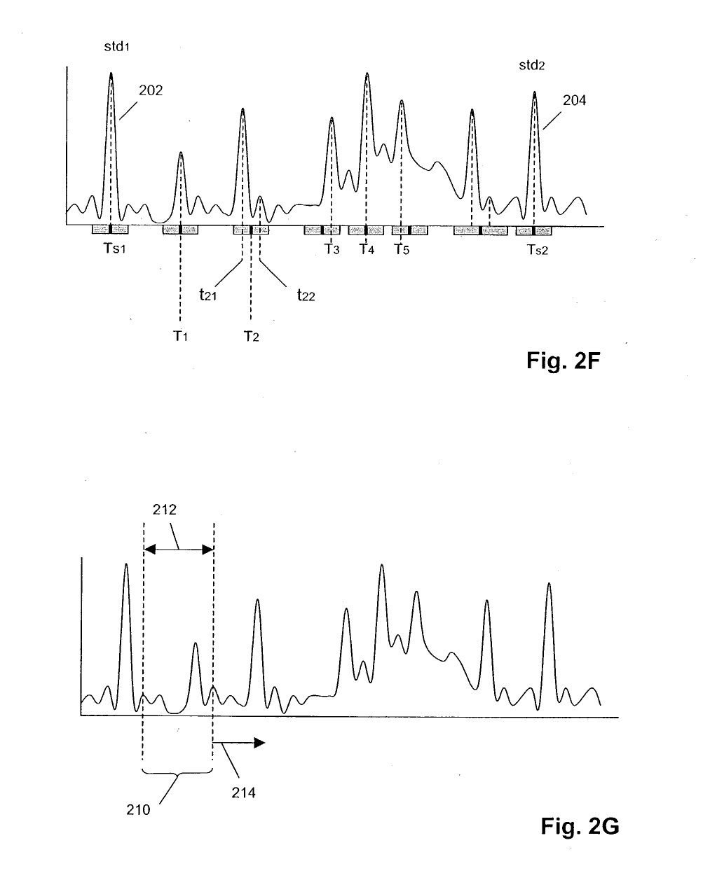

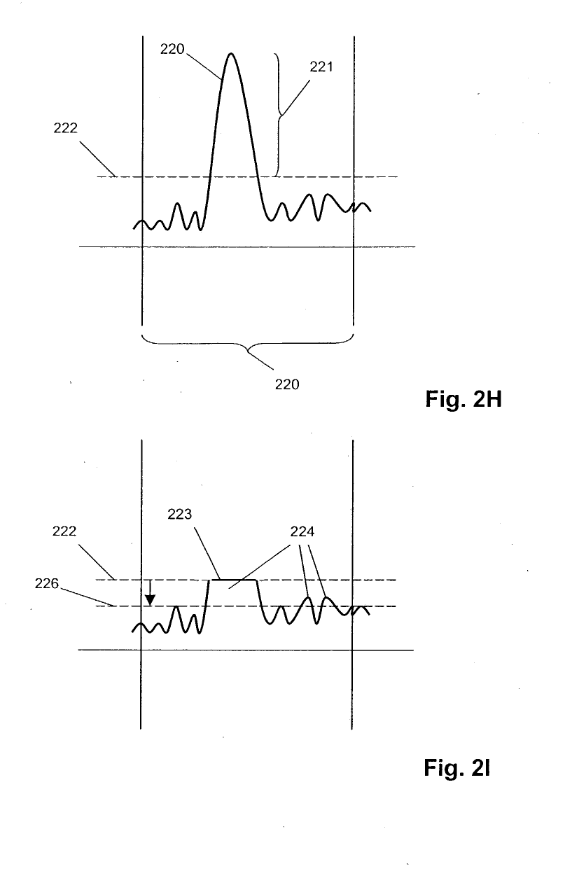

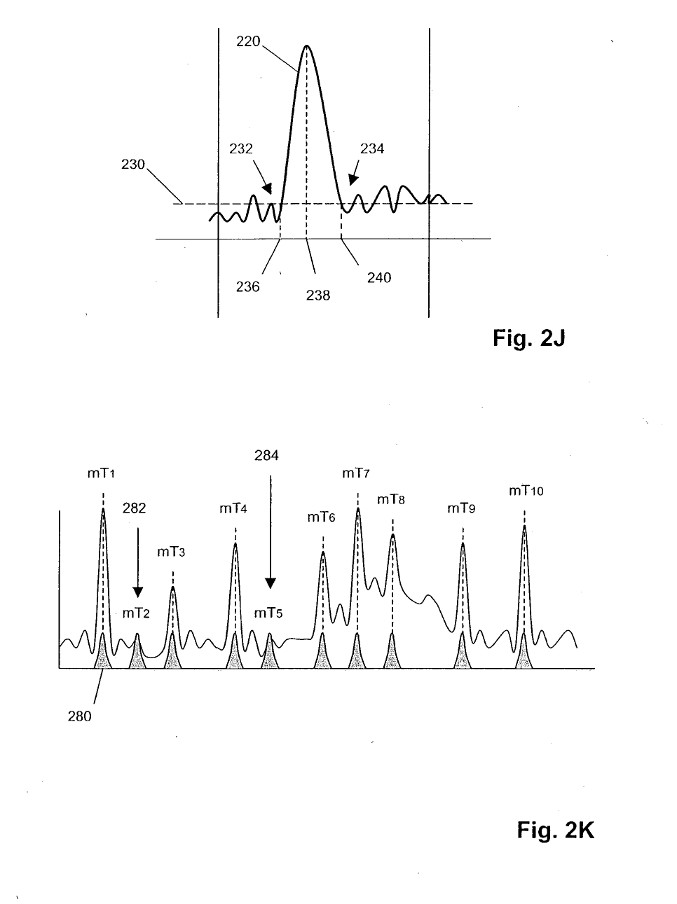

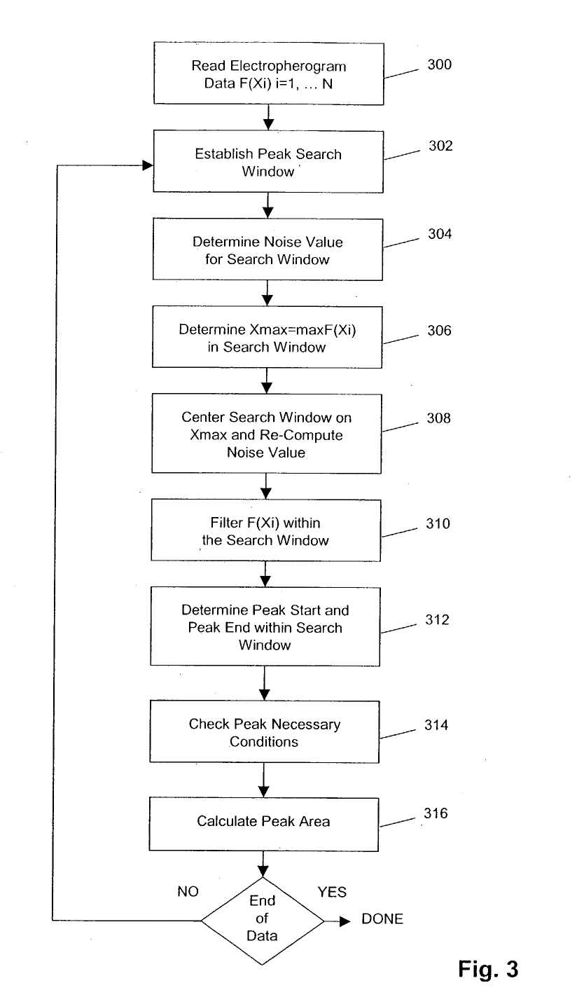

A typical electropherogram (200) displaying electropherogram data is illustrated in FIG. 2A. Several peaks are shown, including a first electrophorectic standard (202) (“std₁”), peaks corresponding to molecular tags mT₁ through mT₆, and a second electrophorectic standard 9204 (“std₂”). Factors that complicate the identification, or correlation, of peaks with molecular tags include noise (205) that may be time dependent, variability between adjacent peaks, or stretching or compressions (208), elevation or variability in the “baseline” signal (206), and the like. As explain more fully below, an object of the present invention is to provide methods for accurately correlating peaks in electropherogram data with molecular tags in view of the above-mentioned distortions in the data. As illustrated in FIG. 2B, in one aspect, the invention provides measures of peak locations relative to the positions of one or more electrophorctic standards. In particular, a migration time T₃ (252) for a molecular tag, “mT₃”, is provided as the following ratio:

### T₃=(t₃-Tₛ₁)/(Tₛ₂-Tₛ₁) where t3 is the observed migration time and Tₛ₁ and Tₛ₂ are the migration times of electrophorectic standards (202) and (204), respectively.

The method of correlating peaks in electropherogram data with molecular tags follows the general steps in FIG. 2C. After electropherogram data is read (290) by a processing unit, peak locations are identified (292) and peak sizes are determined (294). Finally, all or a subset of identified peaks are correlated (296) with molecular tags used in the assay. Preferably, peak size is correlated to the amount of analyte in a sample. A variety of measures may be used for peak size, including peak height, peak area, or the like. Preferably, peak area is used as a measure of peak size. Peak area may estimated is a variety of ways, including taking the product of peak height and peak width at half maximum height, curve fitting, intergration of time averaged values of signal height, and the like.

In one aspect of the invention, two electrophorectic standards are employed, a first electrophoretic standard, e.g. (202) in FIG. 2A, and a second electrophoretic standard, e.g. (204) in FIG. 2A. All other molecular tags used in an assay are selected so that their peaks in electropherogram data falls between the first electrophoretic standard and the second electrophoretic standard, e.g. as illustrated by molecular tags, “mT₁”, “mT₂”, “mT₃”, “mT₄”, “mT₅”, and “mT₆”, shown in FIG. 2A. In other embodiments, more than two electrophoretic standards may be used and the locations of the standards may be among the peaks corresponding to molecular tags, and not necessarily before and after the locations of such peaks.

In another aspect of the invention, makes use of the fact that molecular tags are designed to have either predetermined electrophoretic mobilities and optical properties. If sufficient numbers of a particular tag are released in an assay, then that molecular tag may itself serve as an electrophoretic standard for identification of subsequent peaks. This is advantageous because the closer the reference peak or standard is to a peak whose location is being determined, the more accurate the value for the peak location. As used herein, the term “qualified peak” refers to a peak in electropherogram data that is correlated to a particular molecular tag and that fulfills predetermined criteria for use as an electrophoretic standard. Such criteria may include a measure for peak signal-to-noise ratio, absolute peak height, peak width, or the like. Preferably, a peak is a qualified peak if the peak signal-to-noise ratio is greater than or equal to 1.5; and more preferably, 2.0; and still more preferably, 2.5. In this embodiment, the accuracy of peak identification may vary according to the presence or absence of analytes in a sample because all, some, or none of the molecular tags may be released in detectable amounts, thereby giving rise to a greater or lesser number of available standards.

In another aspect of the invention, illustrated in FIG. 2K, each molecular tag serves as its own standard for identifying peak locations. The figure shows an electropherogram having ten peaks, mT_{1,} through mT₁₀. Each of the peaks comprises signal contributions from molecular tags released in the assay and molecular tag standards (280). As shown with molecular tags, mT₂ (282) and mT₅ (284), when no molecular tag is released in the assay, then the observed peak is entirely due to the standard, which is present in a known and detectable quantity.

Several types of assays may be employed for generating molecular tags that are analyzed in accordance with the invention, such as those exemplified in FIGS. 1A-1C. In FIG. 1A, the Kth analyte (1000) in a plurality of n analytes in a sample is bound by first binding agent (1002), an antibody in this case, having cleavage-inducing moiety (1006) attached, which in this case is a photosensitizer. Photosensitizer (1006) has an effective proximity (1008) within which singlet oxygen generated by it upon photoactivation can cleave the cleavable linkages holding molecular tags (“Tₖ”) (1010) onto second binding agent (1004). After photoactivation (1009), molecular tags within effective proximity (1008) are released along with molecular tags from other binding complexes to form mixture (1012), which is introduced (1014) into a electrophoretic separation apparatus and separated into distinct bands (1016). Separated tags are detected using conventional detection methodologies. For example, if the molecular tags carry fluorescent labels, then detection occurs after illumination by light source (1020) and collection of fluorescence by detector (1018). Detectable product (1016) is then detected at a detection station as described for FIG. 1A.

In FIG. 1B, a method of generating molecular tags is illustrated that is based on a “taqman” polymerase chain reaction (PCR). While target polynucleotide (1030) is amplified by PCR using primers (1032) and (1034), binding compound (1036) specifically hybridizes (1040) to one strand of the target polynucleotide during primer extension and is degraded by the 5′→3′ exonuclease activity of DNA polymerase (1038), resulting (1042) in the release of molecular tag (1044) (shown as “D-M-N”). After several cycles (1046), sufficient molecular tag is released to generate a detectable signal after electrophoretic separation. In FIG. 1C, a method of generating molecular tags is illustrated that is based on an “Invader” reaction. Invader probe (1052) and detection probe (1054) specifically hybridize to target polynucleotide (1050) and form a structure that is recognized by a cleavase (1056), after which the nuclease activity of the cleavase releases molecular tag (1058) leaving cleaved detection probe (1054) is selected so that there is rapid replacement (1062) of cleaved detection probe (1060) with uncleaved detection probe (1064), which is present in excess. As above, reaction cycles continue (1066) until sufficient molecular tag is released to generate a detectable signal after electrophoretic separation.

Samples containing analytes may come from a wide variety of sources including cell cultures, animal or plant tissues, microoraganisms, or the like. Samples are prepared for assays of the invention using conventional techniques, which may depend on the source from which a sample is taken. Guidance for sample preparation techniques can be found in standard treatises, such as Sambrook et al, Molecular Cloning, Second Edition (Cold Spring Harbor Laboratory Press, New York, 1989); Innis et al, editors, PCR Protocols (Academic Press, New York, 1990); Berger and Kimmel, “Guide to Molecular Cloning Techniques,” Vol. 152, Methods in Enzymology (Academic Press, New York, 1987); Ohlendieck, K (1996). Protein Purification Protocols; Methods in Molecular Biology, Humana Press Inc., Totowa N..J. Vol 59:293-304; Method Booklet 5, “Signal Transduction” (Biosource International, Camarillo, Calif., 2002); or the like. For mammalian tissue culture cells, or the like sources, samples containing analytes may be prepared by conventional cell lysis techniques (e.g. 0.14 M NaC1, 1.5 mM MgC1₂, 10 mM Tris-C1 (pH 8.6), 0.5% Nonidet P-40, and protease and/or phosphates inhibitors as required).

In one aspect of the present invention, sets of molecular tags are released fron binding compounds. Molecular tags within a set may be chemically diverse; however, for convenience, sets of molecular tags are usually chemically related. For example, they may all be peptides, or they may consist of different combinations of the same basic building blocks or monomers, or they may be synthesized using the same basic scaffold with different substituent groups for imparting different separation characteristics, as described more fully below. The number of molecular tags in a plurality may vary depending on several factors including the mode separation employed, the labels used on the molecular tags for detection, the sensitivity of the binding moieties, the efficiency with which the cleavable linkages are cleaved, and the like. In one aspect, the number of molecular tags in a plurality ranges from 2 to several tens, e.g. 50. In other aspects, the size of the plurality may be in the range of from 2 to 40, 2 to 20, 2 to 10, 3 to 50, 3 to 20, 3 to 10, 4 to 50, 4 to 10, 5 to 20, or 5 to 10.

### Binding Compounds and Molecular Tags

An aspect of the invention includes providing mixtures of pluralities of different binding compounds, wherein each different binding compound has one or more molecular tags attached through cleavable linkages. The nature of the binding compound, cleavable linkage and molecular tag may vary widely. A binding compound may comprise a binding moiety, such as an antibody binding composition, an antibody, a peptide, a peptide or non-peptide ligand for a cell surface receptor, a protein, an oligonucleotide, an olgonucleotide analog, such as a peptide nucleic acid, a lectin, or any other molecular entity that is capable of specific binding or complex formation with any analyte of interest. In one aspect, a binding compound, which can be represented by the formula below, comprises one or more molecular tags attached to an analyte-specific binding moiety.

B-(L-E)ₖ

wherein B is a binding moiety; L is a cleavable linkage; and E is a molecular tag. Preferably, in homogeneous assays for non-polynucleotide analytes, cleavable linkages, L, is an oxidation-labile linkage, and more preferably, it is a linkage that may be cleaved by singlet oxygen. The moiety “-(L-E)ₖ” indicates that a single binding compound may have multiple molecular tags attached via cleavable linkages. In one aspect, k is an interger greater than or equal to one, but in other embodiments, k may be greater than several hundred, e.g. 100 to 500, or k is greater than several hundred to as many as several thousand, e.g. 500 to 5000. Within a composition of the invention, usually each of the plurality of different types of binding compound has a different molecular tag E. Cleavable linkages, e.g. oxidation-liable linkages, and molecular tags, E, are attached to B by way of conventional chemistries.

Once each of the binding compounds is seperately conjugated with a different molecular tag, it is pooled with other binding compounds to form a plurality of binding compounds, or a binding composition. Usually, each different kind of binding compounds is present in such a composition in the same proportion; however, proportions may be varied as a design choice so that one or a subset of perticular binding compounds are present in greater or lower proportion depending on the desirability or requirements for a particular embodiment or assay. Factors that may affect such design choices include, but are not limited to, antibody affinity and avidity for a particular target, relative prevalence of a target, fluorescent characteristics of a detection moiety of molecular tag, and the like.

In one aspect, B is an oligonucleotide defined by the following formula:

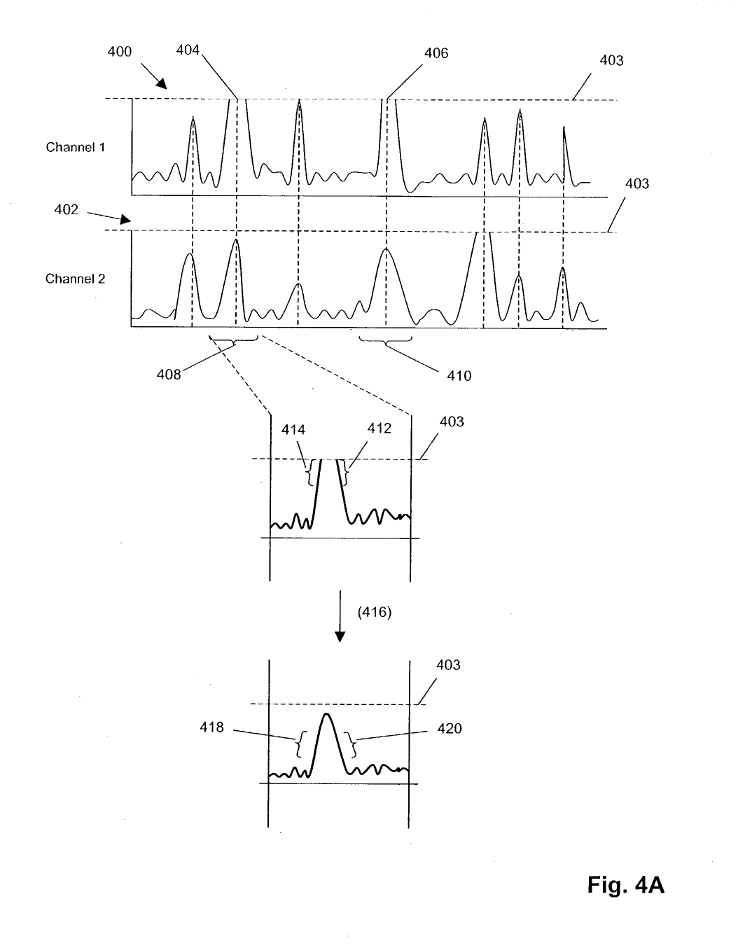

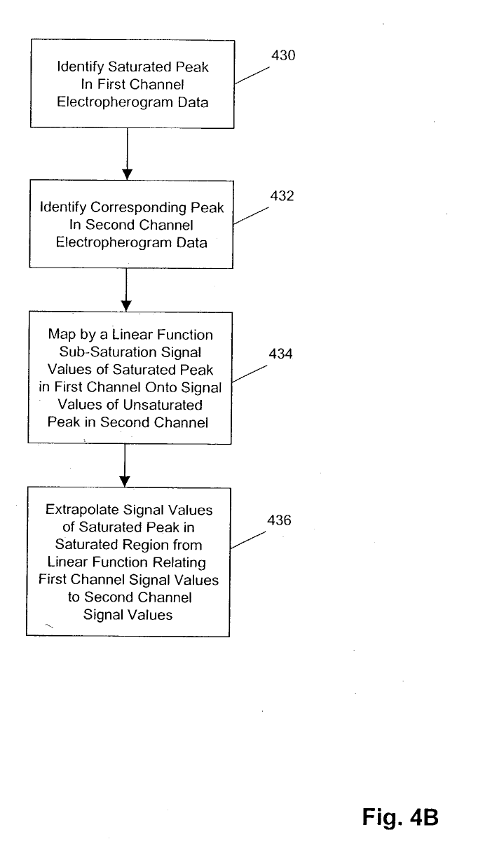

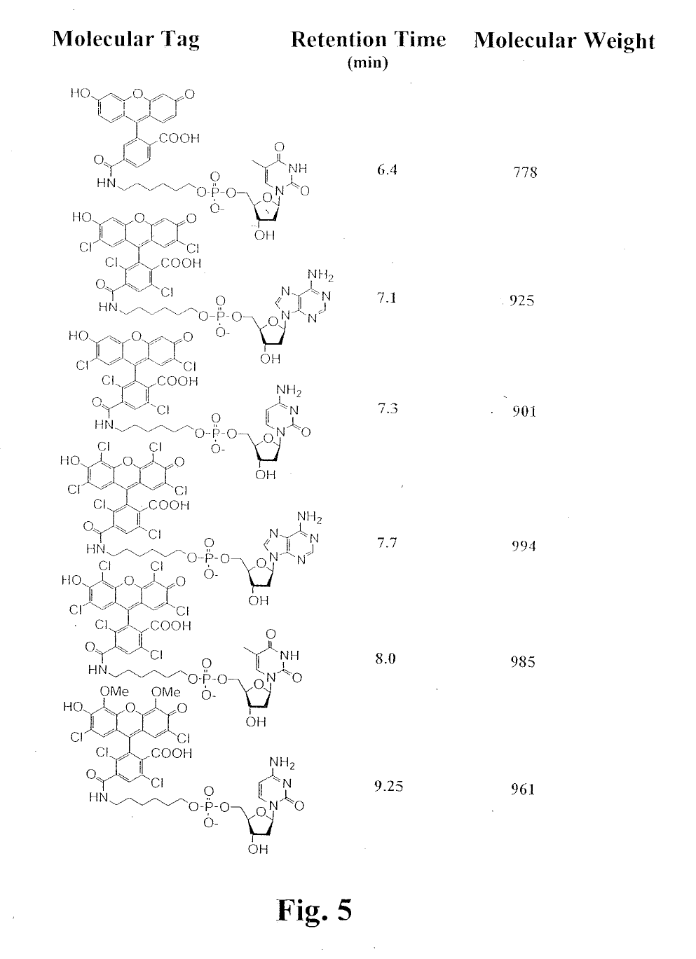

E-N-T where E is as defined above, N is nucleotide, and T is an oligonucleotide specific for a polynucleotide analyte. Preferably, N is attached to the 5′ nucleotide of T by way of a natural phosphodiester bond. E may be attached to N via several different attachments sites, either on the base of N or its ribose or deoxyribose moiety. Preferably, E is attached to the 5′ carbon of N by way of a phosphodiester bond. Synthesis of such compounds is taught in U.S. Pat. Nos. 6,322,980 and 6,514,700, which are incorporatd by reference; and in International patent publication WO 01/83502. In this class of binding compound, the cleavable linkage is preferably the phosphodiester bond between N and T, and it is cleaved by way of an enzymatic reaction by a nuclease that recognizes specific structures formed by the binding compound, the target polynucleotide, and possibly other molecular elements. As a result of the enzymatic reaction molecular tag of the form “E-N” are released. Preferably, the enzymatic reaction is in conjunction with an amplification reaction so that in a single assay each target polynucleotide gives rise to many hundreds, or thousands, of released molecular tags. In one aspect, molecular tags may be generated by any one of several nucleic acid-based signal amplification techniques that use the degradation of a probe with a nuclease activity, including but not limitied to “taqman” assays, e.g. Gelfrand, U.S. Pat. No. 5,210,015; probe-cycling assays, e.g. Brow et al, U.S. Pat. No. 5,846,717; Walder et al, U.S. Pat. No. 5,403,711; Hogan et al, U.S. Pat. No. 5,451,503; Western et al, U.S. Pat. No. 6,121,001; Fritch et al, U.S. Pat. No. 4,725,537; Vary et al U.S. Pat. No. 4,767,699; and other degradation assays, e.g. Okano and Kambara, Anal. Biochem, 228: 101-108 108 (1995). Exemplary released molecular tags of this embodiment are illustrated in FIGS. 4-6. 6. In this embodiment, released molecular tags preferably have the form “(M,D)-N”, where the moiety “(M,D)” is defined as described below.

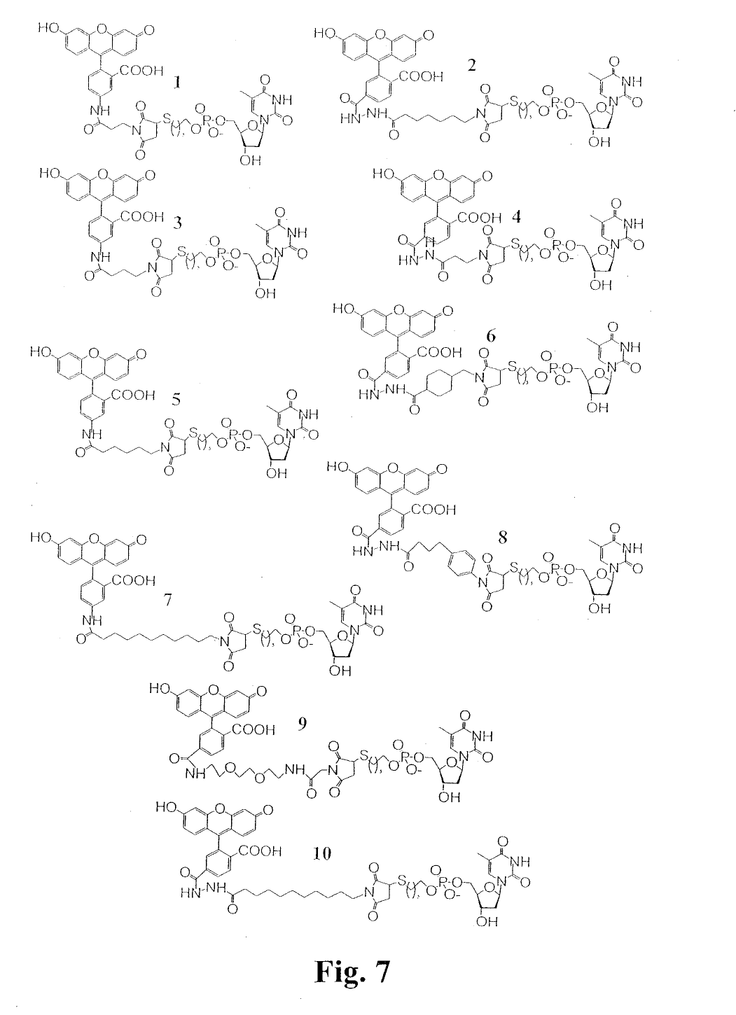

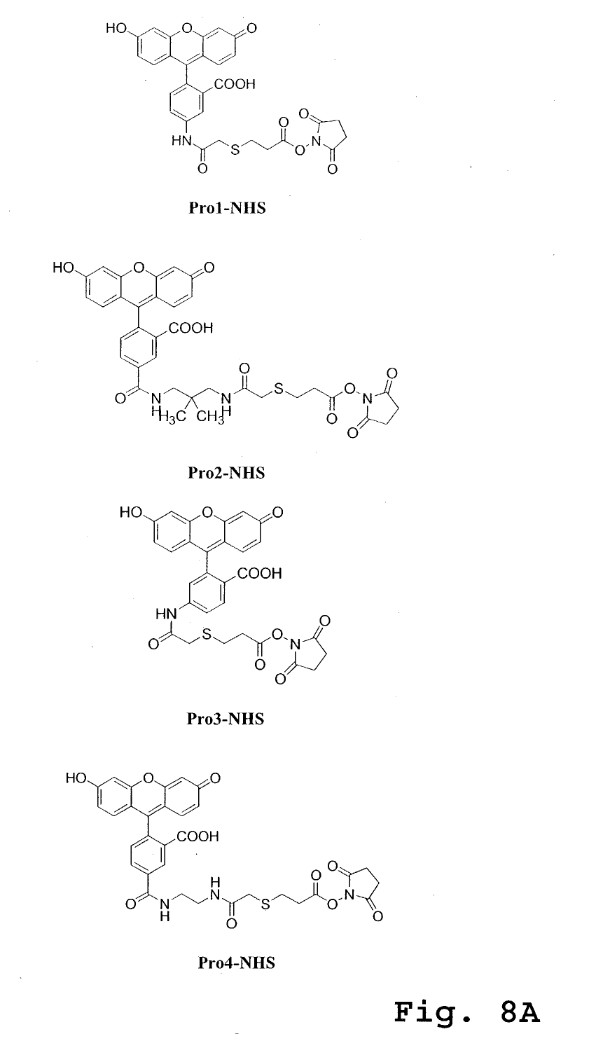

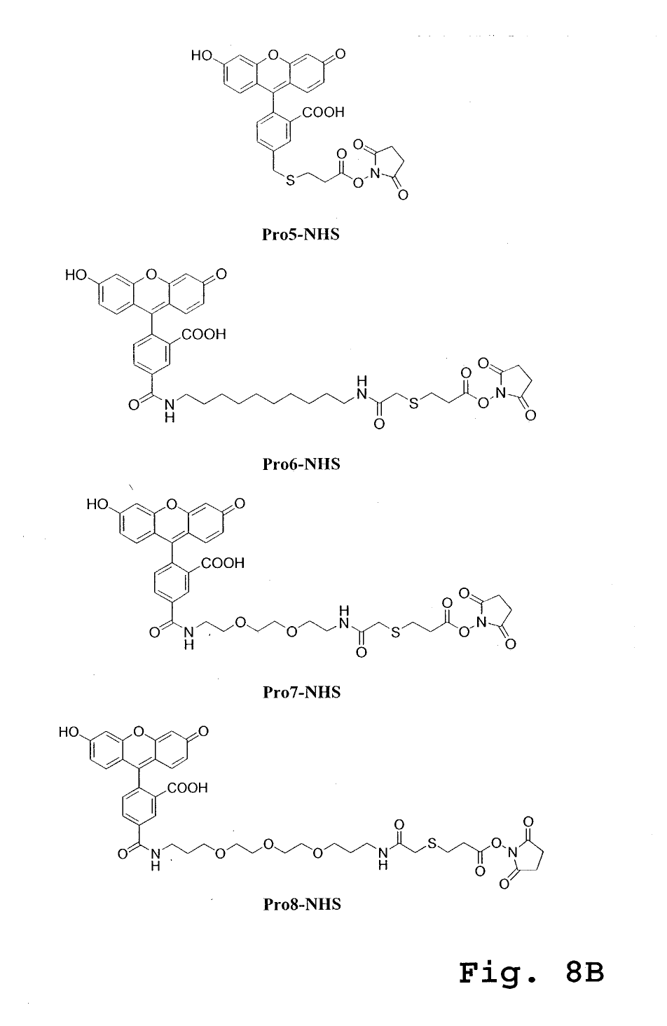

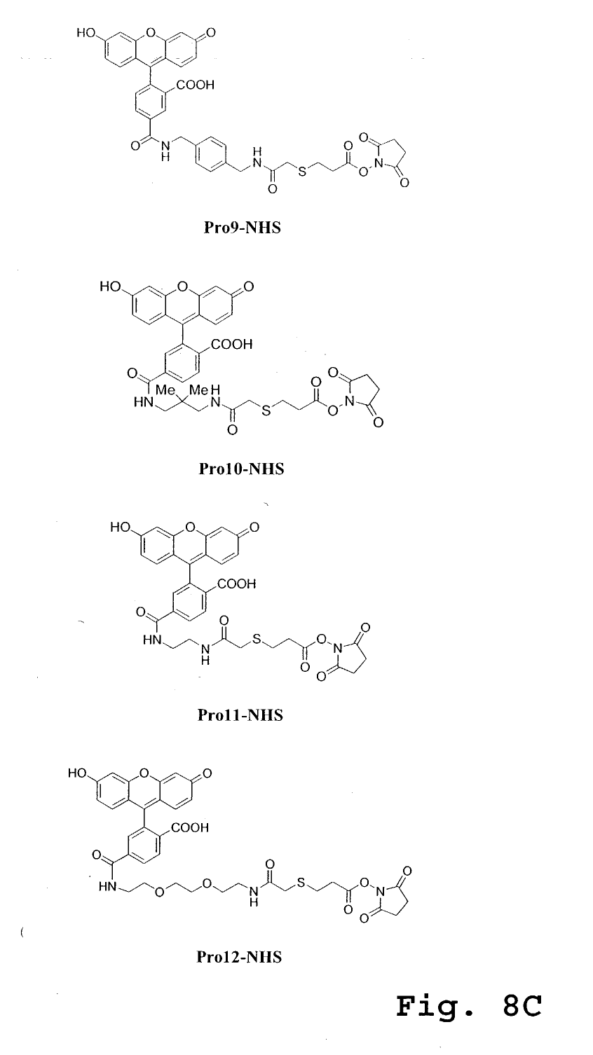

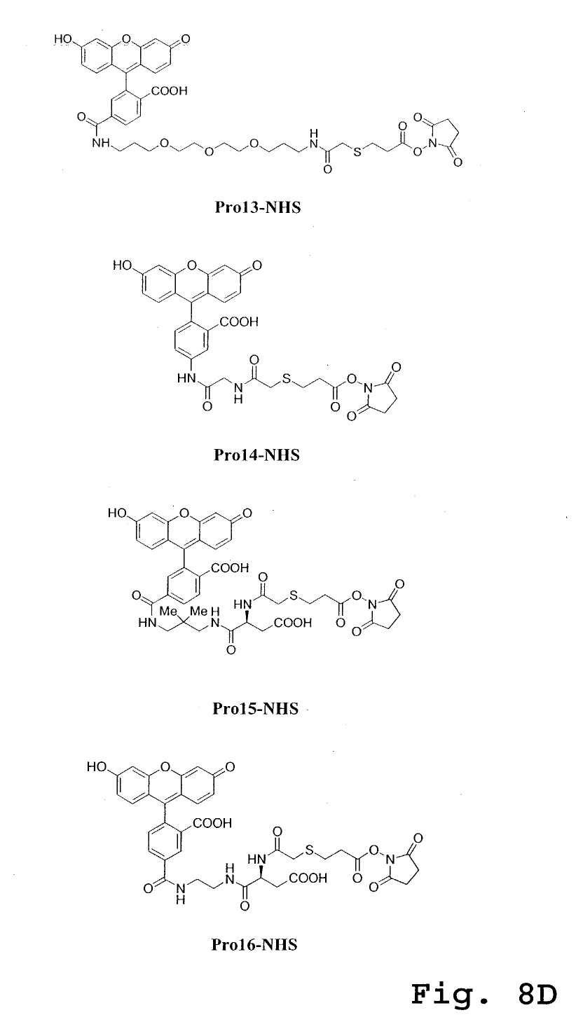

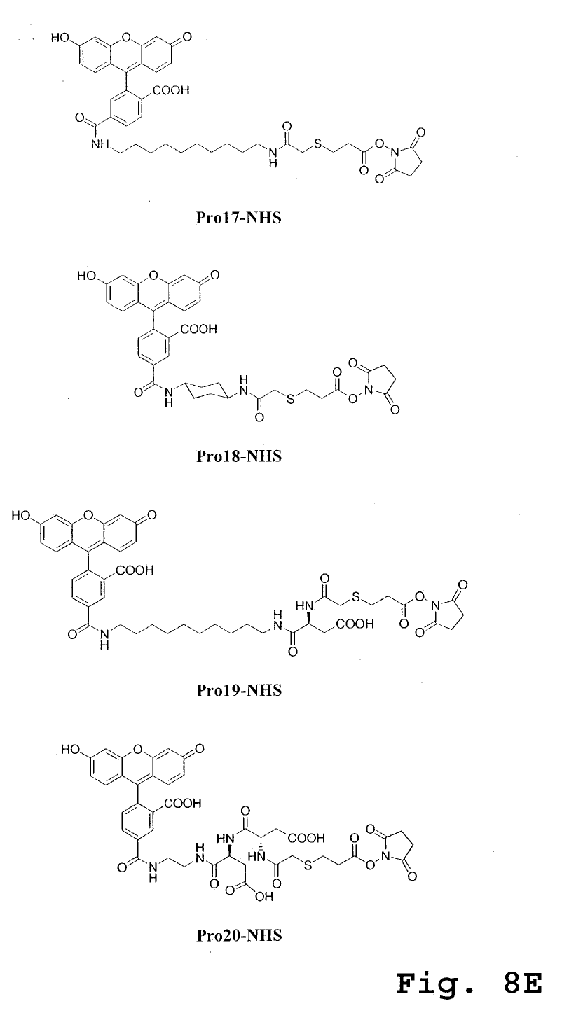

In another aspect, B is an antibody binding composition. Such compositions are readily formed from a wide variety of commercially available antibodies, both monoclonal and polyclonal, specific for a wide variety of analytes. Extensive guidance can be found in the literature for covalently linking molecular tags to binding compounds, such as antibodies, e.g. Hermanson, Bioconjugate Techniques, (Academic Press, New York, 1996), and the like. In one aspect of the invention, one or more molecular tags are attached directly or indirectly to common reactive groups on a binding compound. Common reactive groups include amine, thiol, carboxylate, hydroxyl, aldehyde, ketone, and the like, and may be coupled to molecular tags by commercially available cross-linking agents, e.g. Hermanson (cited above); Haugland, Handbook of Fluorescent Probes and Research Products, Ninth Edition (Molecular Probes, Eugene, Oreg., 2002). In one embodiment, an NHS-ester of a molecular tag is reacted with a free amine on the binding compound. Exemplary NHS-esters of molecular tags suitable for attachment to free amines of binding compounds are shown in FIG. 7A-7J.

When L is oxidation labile, L is preferably a thioether or its selenium analog; or an olefin, which contains carbon-carbon double bonds, wherein cleavage of a double bond to an oxo group, releases the molecular tag, E. Illustrative thioether bonds are disclosed in Willner et al, U.S. Pat. No. 5,622,929 which is incorporated by reference. Illustrative olefins include vinyl sulfides, vinyl ethers, enamines, imines substituted at the carbon atoms with an α-methine (CH, a carbon atom having at least one hydrogen atom), where the vinyl group may be in a ring, the heteroatom may be in a ring, or substituted on the cyclic olefinic carbon atom, and there will be at least one and up to four heteroatoms bonded to the olefinic carbon atoms. The resulting dioxetane may decompose spontaneously, by heating above ambient temperature, usually below about 75° C., by reaction with acid or base, or by photo-activation in the absence or presence of a photosensitizer. Such reactions are described in the following exemplary references: Adam and Liu, J. Amer. Chem. Soc. 94, 1206-1209, 1972, Ando, et al., J.C.S. Chem. Comm. 1972, 477-8, Ando, et al., Tetrahedron 29, 1507-13, 1973, Ando, et al., J. Amer. Chem. Soc. 96, 6766-8, 1974, Ando and Migita, ibid. 97, 5028-9, 1975, Wasserman and Terao, Tetra. Lett. 21, 1735-38, 1975, Ando and Watanabe, ibid. 47, 4127-30, 1975, Zaklika, et al., Photochemistry and Photobiology 30,35-44, 1979, and Adam, et al.,Tetra. Lett. 36, 78534, 1995. See also, U.S. Pat. No. 5,756,726.

The formation of dioxetanes is obtained by the reaction of singlet oxygen with an activated olefin substituted with a molecular tag at one carbon atom and the binding moiety at the other carbon atom of the olefin. See, for example, U.S. Pat. No. 5,807,675 and International patent publication WO 01/83502; which are incorporated by reference.

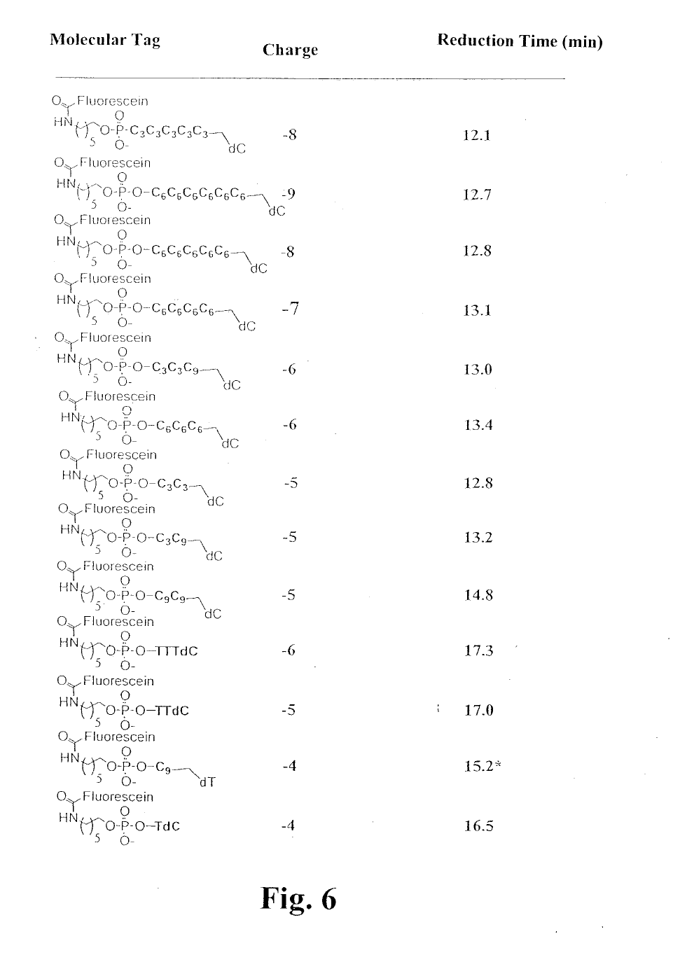

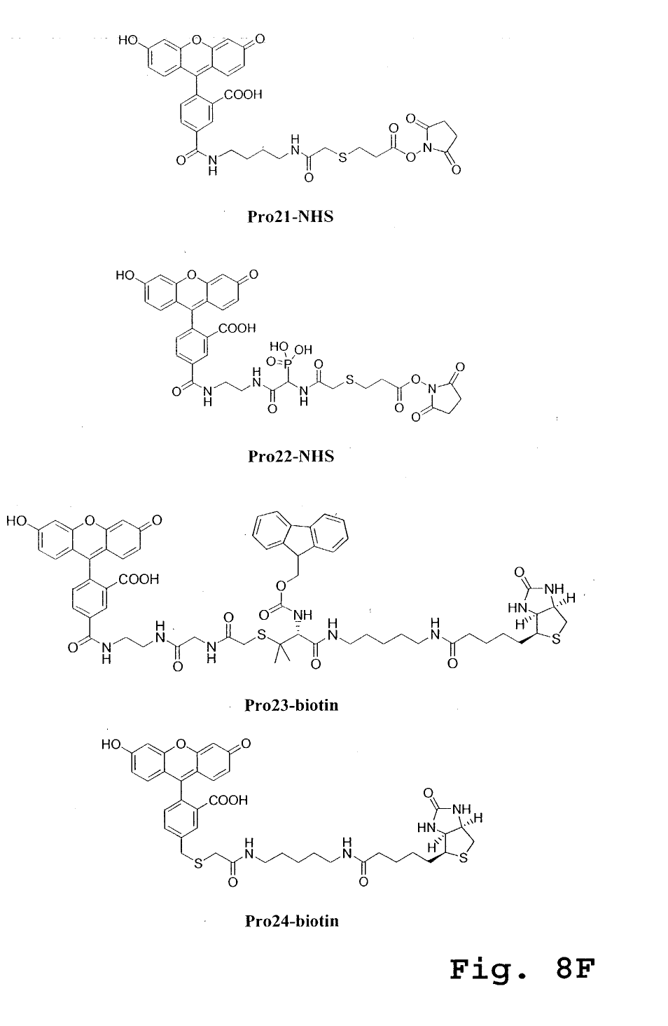

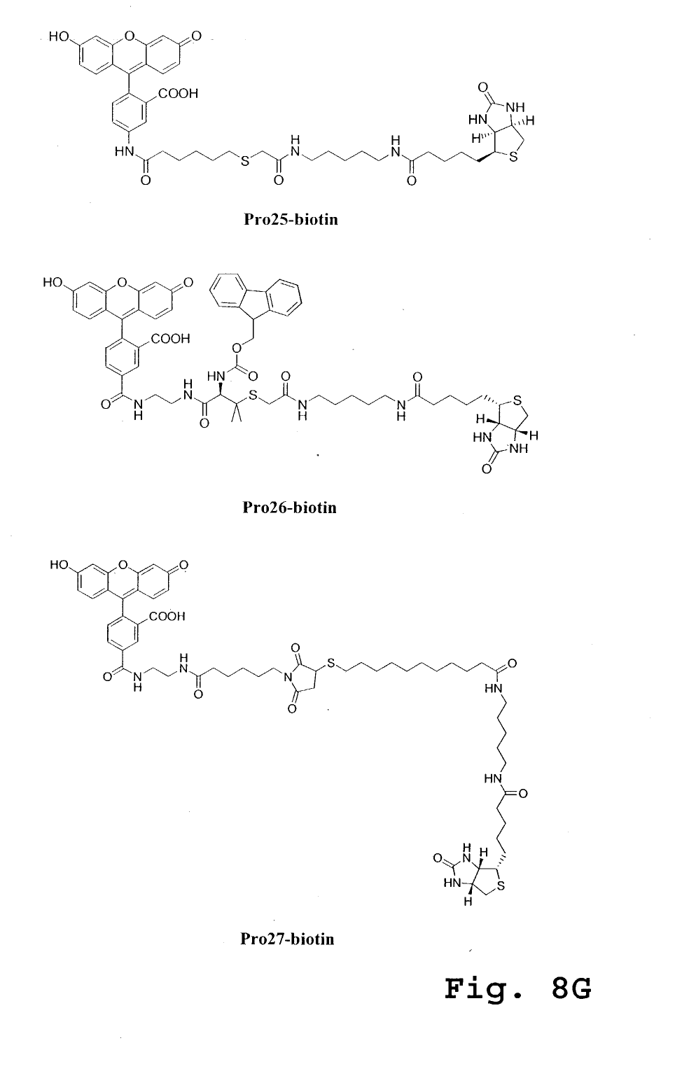

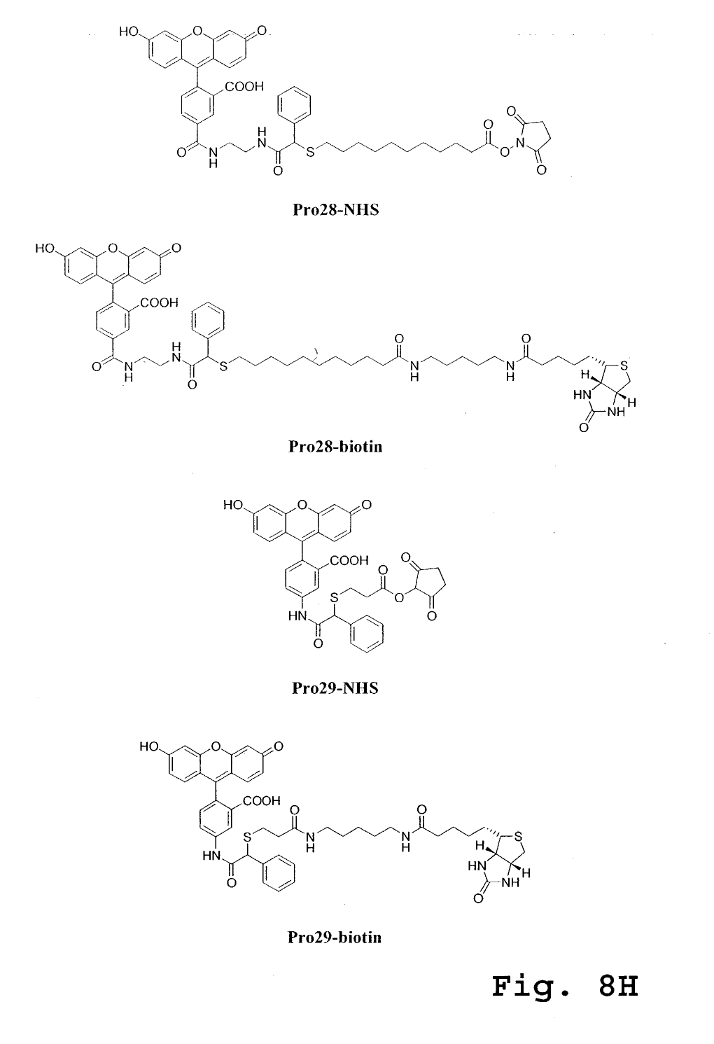

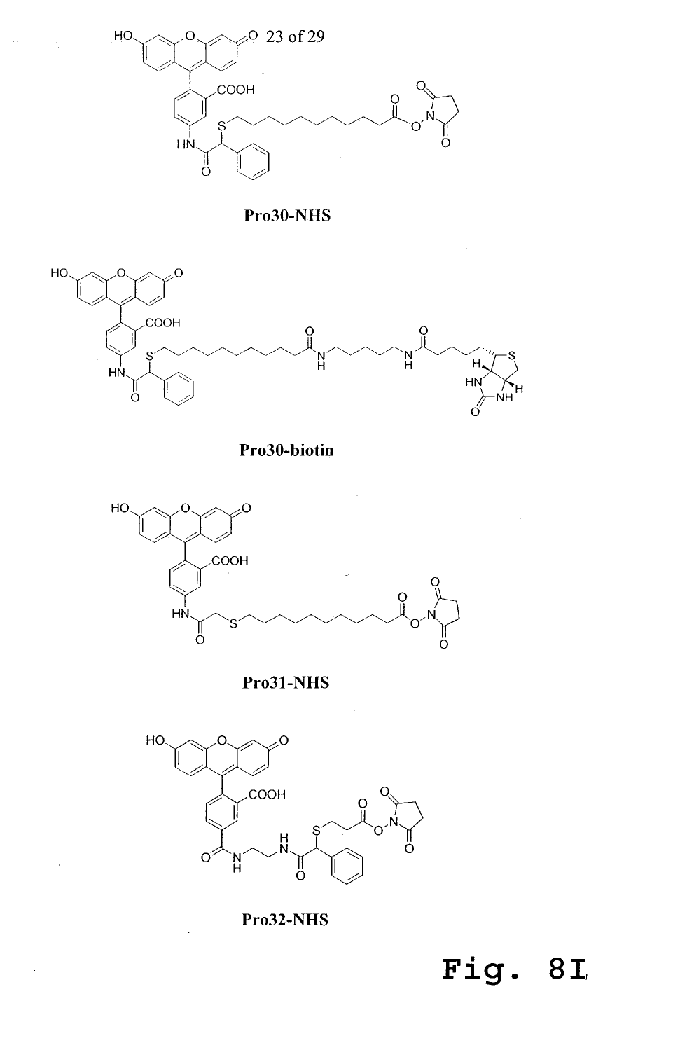

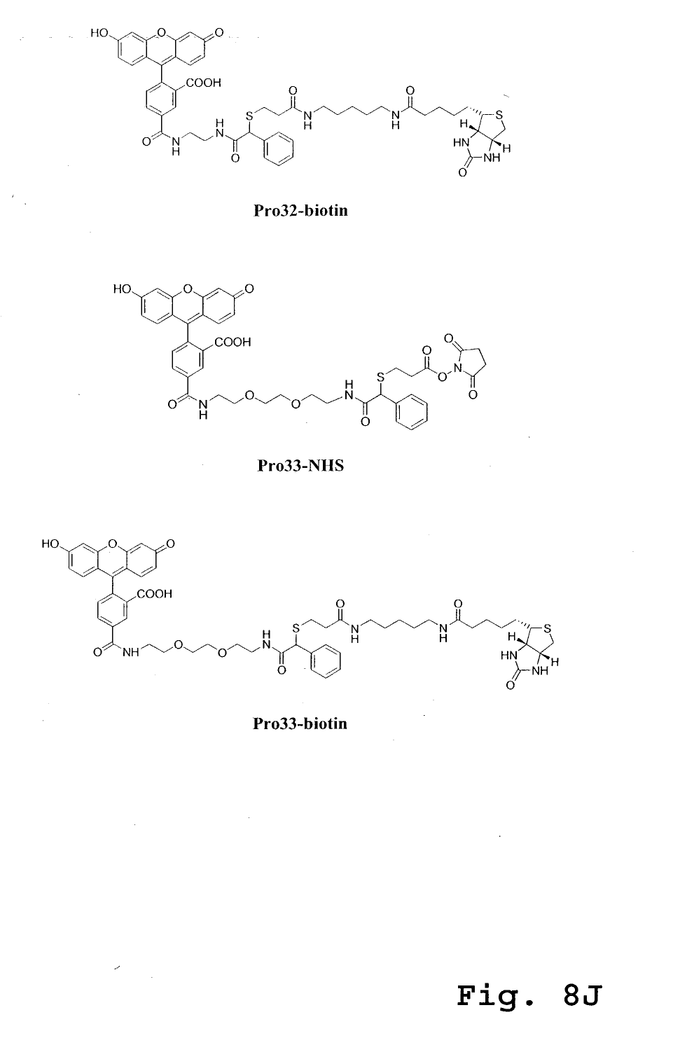

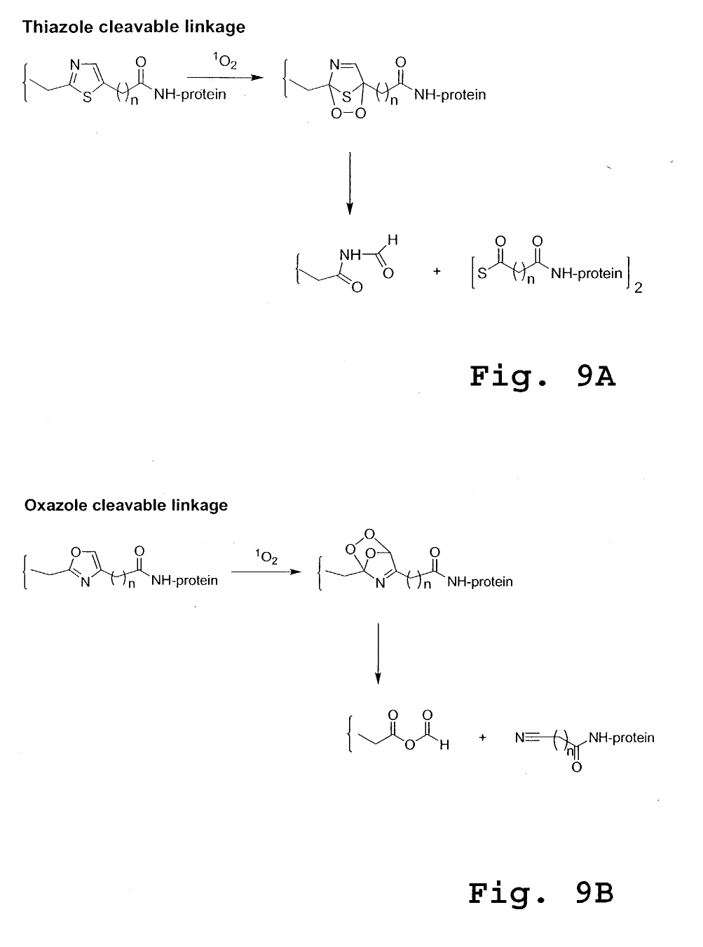

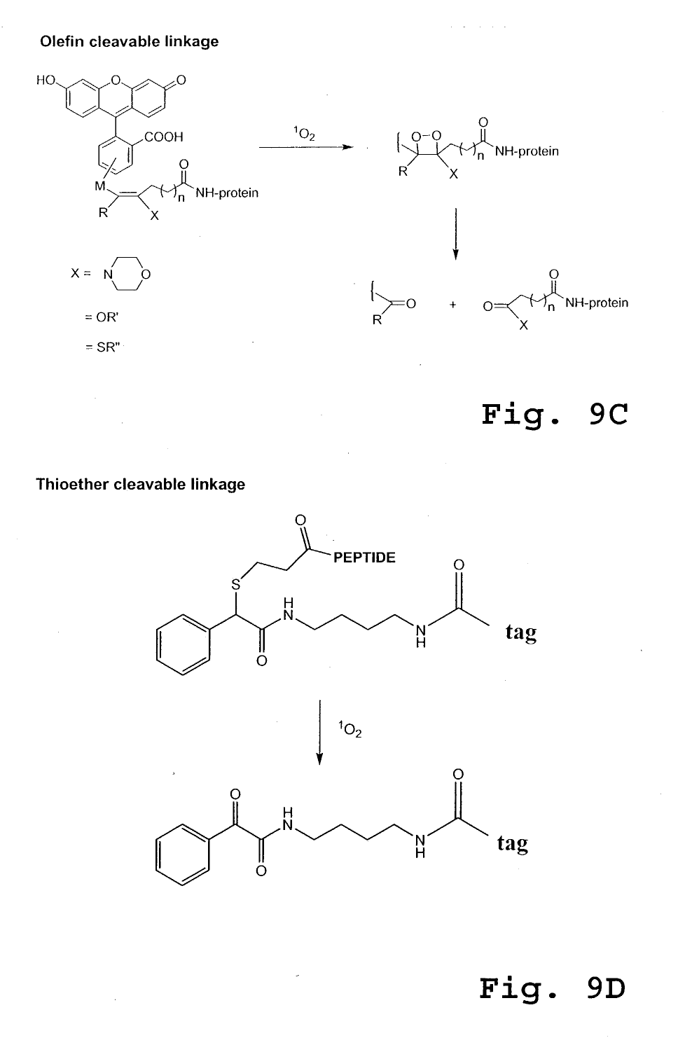

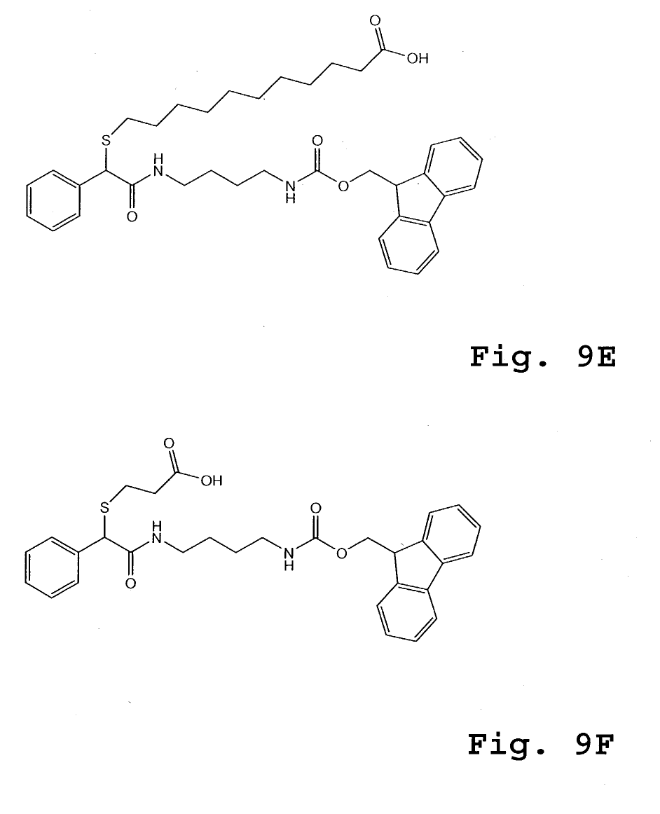

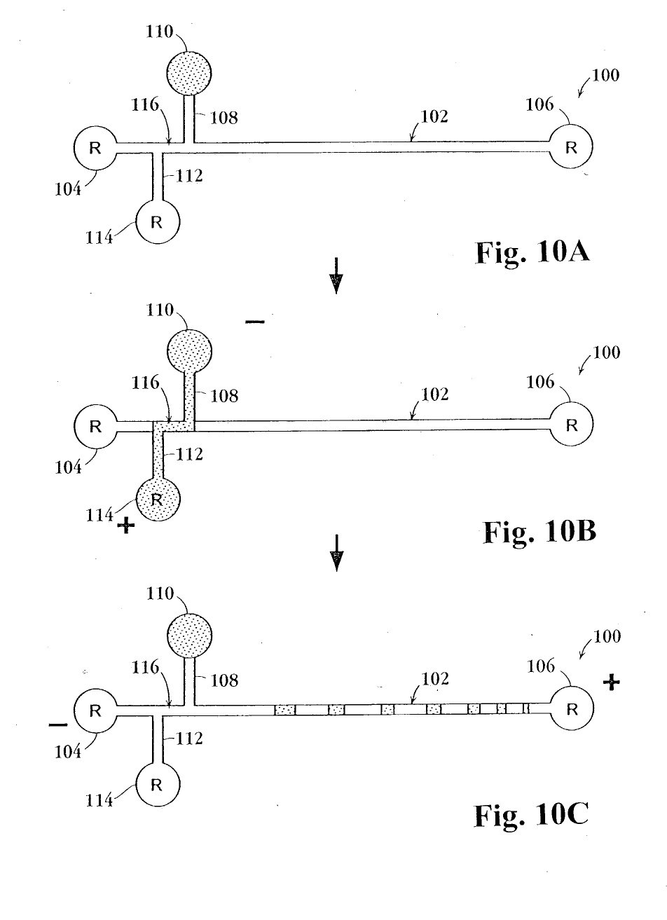

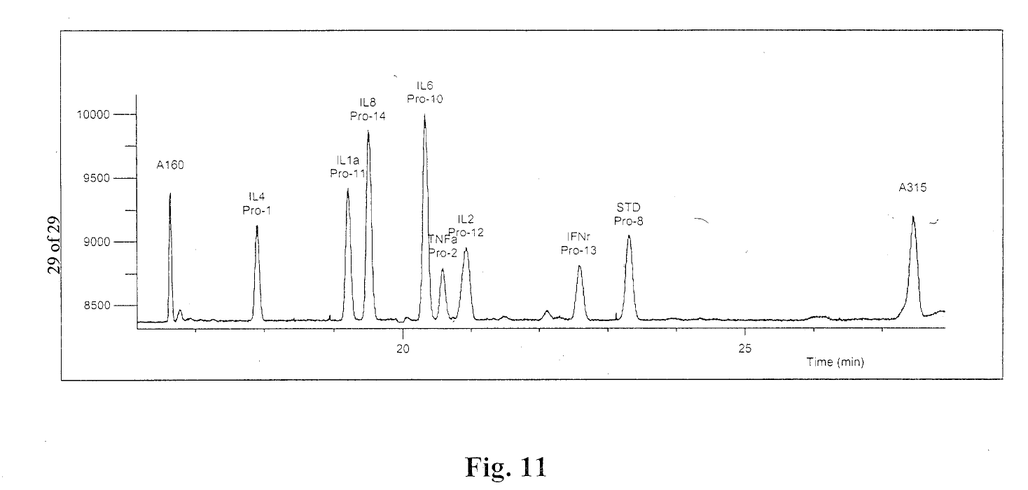

Several cleavable linkages and their cleavage products are illustrated in FIGS. 8A-F. The thiazole cleavable linkage,“-CH₂-thiazole-(CH2)ₙ-C(=O)-NH-protein,” shown in FIG. 8A, results in an molecular tag with the moiety “-CH2-C(=O)-NH-CHO.” Preferably, n is in the range of from 1 to 12, and more preferably, from 1 to 6. The oxazole cleavable linkage, “-CH₂-oxazole oxazole-(CH2)ₙ-C(=O)-NH protein,” shown in FIG. 8B, results in an molecular tag with the moiety “-CH₂-C(=O)O-CHO.” An olefin cleavable linkage (FIG. 8C) is shown in connection with the binding compound embodiment “B-L-M-D,” described above and with D being a fluorescein dye. The olefin cleavable linkage may be employed in other embodiments also. Cleavage of the illustrated olefin linkage results in an molecular tag of the form: “R-(C=O)-M-D,” where “R” may be any substituent within the general description of the molecular tags, E, provided above. Preferably, R is an electron-donating group, e.g. Ullman et al, U.S. Pat. No. 6,251,581; Smith and March, March's Advanced Organic chemistry: Reactions, Mechanisms, and Structure, 5^{th} Edition (Wiley-Interscience, New York, 2001); and the like. More preferably, R is an electron-donating group having from 1-8 carbon atoms and from 0 to 4 heteroatoms selected from the group consisting of O, S, and N. In further preference, R is -N(Q)₂, -OQ, p- [C₆H₄N(Q)₂], furanyl, n-alkylpyrrolyl, 2-indolyl, or the like, where Q is alkyl or aryl. In further reference to the olefin cleavable linkage of FIG. 8C, substituents “X” and “R” are equivalent to substituents “X” and “Y” of the above formula describing cleavable linkage, L. In particular, X in FIG. 8C is preferably morpholino, -OR′, or -SR″, where R′ and R″ are aliphatic, aromatic, alicyclic or heterocyclic having from 1 to 8 carbon atoms and 0 to 4 heteroatoms selected from the group consisting of O, S. and N. A preferred thiother cleavable linkage is illustrated in FIG. 6D having the form “-(CH₂)₂-S-CH(C₆H₅)C(=O)NH-(CH₂)ₙ-NH-,” wherein n is in the range of from 2 to 12, and more preferebly, in the range of from 2 to 6. Thiother cleavable linkages of the type shown in FIG. 8D may be attached to binding moieties, T, and molecular tags, E, by way of precursor compounds shown in FIG. 8e and 8f. To attach to an amino group of a binding moiety, T, the terminal hydroxyl is converted to an NHS ester by conventional chemistry. After reaction with the amino group and attachment, the Fmoc protection group is removed to produce a free amine which is then reacted with an NHS ester fo the molecular tag, such as compounds produced by the schemes of FIGS. 1,2,and 4, with the exception that the last reaction step is the addition of an NHS ester, instead of a phosphoramidite group. Molecular tag E, is preferably a water-soluble organic compound that is stable with respect tot he active species, especially singlet oxygen, and that includes a detection or reporter group. Otherwise, E may vary widely in size and structure. In one aspect, E has a moleculaar weight in the range of from about 50 to 2500 daltons, more preferably, from about 50 to about 1500 daltons. Preferred structures of E are described more fully below. E may comprise a detection group fro generating an electrochemical, fluorescent, or chromogenic signal. Preferably, the detection group generates a fluorescent signal. Electrophoretic standards of the invention may be selected from the same set of compounds as are the molecular tag. In one aspect, one or more molecular tags in a plurality may be designated and used as electrophoretic standards in the method of the invention. When used as an electrophoretic standard, a known quantity of the molecular tag is added to the mixture to be separated, That is, molecular tags used as electrophoretic standards are not released from binding compound, they are prepared in their released form and added directly to the mixture to be separated.

Molecular tags within a plurality are selected so that each has a unique electrophoretic separation characteristic and/or a unique optical property with respect to the other memebers of the same plurality. In one aspect, the electrophoretic separation charachteristics is migration time under set of standard separation conditions conventional in the art, e.g. voltage, capillary type, electrophoretic separation medium, or the like. In another aspect, the optical property is a fluorescence property, such as emission spectrum, fluorescence lifetime, fluorescence intensity at a given wavelength or band of wavelengths, or the like. Preferably, the fluorescence property is fluorescence intensity. For example, each molecular tag of a plurality may have the same fluorescent emission properties, but each will differ from one another by virtue of a unique migration time. On the other hand, or two or more of the molecular tags of a plurality may have identical migration times, but will have unique fluorescent properties, e.g. spectrally resolvable emission spectra, so that all the members of the plurality are distinguished by the combination of molecular separation and fluorenscence measurment.

Preferably, released molecular tags are detected by electrophoretic separation and the fluorescence of a detection group. In such embodiments, molecular tags having substantially identical fluorescence properties have different electrophoretic mobilities so that distinct peaks in an electropherogram are formed under separation conditions. Preferably, pluralities of molecular tags of the invention are separated by conventional capillary electrophoresis apparatus, either in the presence or absence of a conventional sieving matrix. Exemplary capillary electrophoresis apparatus include Applied biosystems (Foster City, Calif.) models 310, 3100 and 3700; Beckman (Fullerton, Calif.) model P/ACE MDQ; Amersham biosciences (Sunnyvale, Calif.) MegaBACE 1000 or 4000; SpectruMedix genetic analysis system; and the like. Electrophoretic mobility is proportional to q/M^{⅔}, where q is the charge on the molecule and M is the mass of the molecule. Desirably, the difference in mobility under the conditions of the determination between the closest electrophoretic labels will be at least about 0.001, usually 0.002, more usually at least about 0.01, and may be 0.02 or more. Preferably, in such conventional apparatus, the electrophoretic mobilities of molecular tags of a plurality differ by at least one percent, and more preferably, by at least a percentage in the range of from 1 to 10 percent.

In one aspect, molecular tag, E, is (M,D), where M is a mobility-modifying moiety and D is a detection moiety. The notation “(M,D)” is used to indicate that the ordering of the M and D moieties may be such that either moiety can be adjacent to the cleavable linkage, L. That is, “B-L-(M,D)” designates binding compound of either of two forms: “B-L-M-D” or “B-L-D-M.”

Detection moiety, D, may be fluorescent label or dye, a chromogenic label dye, an electrochemical label, or the like. Preferably, D is a fluorescent dye. Exemplary fluorescent dyes for use with the invention include water-soluble rhodamine dyes, fluorosceins, 4,7-dichlorofluoresceins, benzoxanthene dyes, and energy transfer dyes, disclosed in the following references: Handbook of Molecular Probes and Research Reagents, 8th ed., (Molecular Probes, Eugene, 2002); Lee et al, U.S. Pat. No. 6,191,278; Lee et al, U.S. Pat. No. 6,372,907; Menchen et al; U.S. Pat. No. 6,096,723; and Lee et al, U.S. Pat. No. 5,945,526. More preferably, D is a fluorescein or a fluorescein derivative.

The size and composition of mobility-modifying moiety, M, can vary from a bond to about 100 atoms in a chain, usually not more than about 60 atoms, more usually not more than about 30 atoms, where the atoms are carbon, oxygen, nitrogen, phosphorous, boron and sulfur. Generally, when other than a bond, the mobility-modifying moiety has from about 0 to about 40, more usually from about to 0 to about 30 heteroatoms, which in addition to the heteroatoms indicated above may include halogen or other heteroatom. The total number of atoms other than hydrogen is generally fewer than about 200 atoms, usually fewer than about 100 atoms. Where acid groups are present, depending upon the pH of the medium in which the mobility-modifying moiety is present, various cations may be associated with the acid group. The acids may be organic or inorganic, including carboxyl, thionocarboxyl, thiocarboxyl, hydroxamic, phosphate, phosphite, phosphonate, phosphinate, sulfonate, sulfinate, boronic, nitric, nitrous, etc. For positive charges, substituents include amino (includes ammonium), phosphonium, sulfonium, oxonium, etc., where substituents are generally aliphatic of from about 1-6 carbon atoms, the total number of carbon atoms per heteroatom, usually be less than about 12, usually less than about 9. The side chains include amines, ammonium salts, hydroxyl groups, including phenolic groups, carboxyl groups, esters, amides, phosphates, heterocycles. M may be homo-oligomer or a hetero-oligomer, having different monomers of the same or different chemical characteristics, e.g., nucleotides and amino acids.

In another aspect, (M,D) moieties are constructed from chemical scaffolds used in the generation of combinatorial libraries. For example, the following references described scaffold compound useful in generating diverse mobility modifying moieties: peptoids (PCT Publication No WO 91/19735, Dec. 26, 1991), encoded peptides (PCT Publication WO 93/20242, Oct. 14 1993), random bio-oligomers (PCT Publicatin WO 92/00091, Jan. 9, 1992), benzodiazepines (U.S. Pat. No. 5,288,514), diversomeres such as hydantoins, benzodiazepines and dipeptides (Hobbs DeWitt, S. et al., Proc. Nat. Acad. Scl. U.S.A. 90: 6909-6913 (1993), vinylogous polypeptides (Hagihara et al. J.Amer. Chem. Soc. 114: 6568 (1992)), nonpeptidal peptidomimetics with a Beta-D-Glucose scaffolding (Hirschmann, R. et al, J.Amer. Chem. Soc. 114: 9217-9218 (1992)), analogous organic syntheses of small compound libraries (Chen, C. et al J.Amer. Chem. Soc. 116: 2661(1994)), oligocarbamates (Cho, C. Y. et al. Science 261: 1303(1993)), peptidyl phosphonates (Campbell, D. A. et al, J. Org. Chem. 59:658(1994)); Cheng et al, U.S. Pat. No. 6,245,937; Heizmann et al, “Xanthines as a scaffold for molecular diversity,” Mol. Divers. 2: 171-174 (1997); Pavia et al, Bioorg. Med. Chem., 4: 659-666 (1996); Ostresh et al, U.S. Pat. No. 5,856,107; Gordon, E. M. et al., J. Med. Chem. 37: 1385 (1994); and the like. Preferably, in this aspect, D is a substituent on a scaffold and M is the rest of the scaffold.

M may also comprise polymer chains prepared by known polymer subunit synthesis methods. Methods of forming selected-length polyethylene oxide-containing chains are well known, e.g. Grossman et al, U.S. Pat. No. 5,777,096. It can be appreciated that these methods, which involve coupling of defined-size, multi-subunit polymer units to one another, directly or via linking groups, are applicable to a wide variety of polymers, such as polyethers (e.g., polyethylene oxide and polypropylene oxide), polyesters (e.g., polyglycolic acid, polylactic acid), polypeptides, oligosaccharides, polyurethanes, polyamides, polysulfonamides, polysulfoxides, polyphosphonates, and block copolymers thereof, including polymers composed of units of multiple subunits linked by charged or uncharged linking groups. In addition to homopolymers, the polymer chains used in accordance with the invention include selected-length copolymers, e.g., copolymers of polyethylene oxide units alternating with polypropylene units. As another example, polypeptides of selected lengths and amino acid composition (i.e., containing naturally occurring or man-made amino acid residues), as homopolymers or mixed polymers.

In another aspect, after release, molecular tag, E, is defined by the formula:

### A-M-D

wherein:

A is -C(=O)R, where R is aliphatic, aromatic, alicyclic or heterocyclic having from 1 to 8 carbon atoms and 0 to 4 heteroatoms selected from the group consisting of O. S. and N; -CH2C(=O)-NHi-CHO; -SO2H; -CH2-C(=O)O-CHO; -C(=O)NH-(CH2)n-NH-C(=O)C(=O)-(C6H5),

where n is in the range of from 2 to 12;

D is a detection group, preferably a fluorescent dye; and

M is as described above, with the proviso that the total molecular weight of A-M-D be within the range of from about 100 to about 2500 daltons.

In another aspect, D is a fluorescein and the total molecular weight of A-M-D is in the range of from about 100 to about 1500 daltons.

In one aspect, assays of the invention employ sensitizer compounds to generate an active species, such as singlet oxygen, to cleave the cleavable linkage attaching a molecular tag to a binding compound. An important consideration for a sensitizer and cleavable linkage is that they not be so far from one another that when a binding compound is bound to a membrane-associated analyte the active species generated by the sensitizer diffuses and loses its activity before it can interact with the cleavable linkage. Accordingly, during a cleavage step, a sensitizer preferably is within 1000 mm, preferably 20-100 nm of a bound cleavage-including moiety. This effective range of a cleavage-inducing moiety is referred to herein as its “effective proximity.”

A preferred sensitizer for use with the invention is photosensitizer that generates singlet oxygen from molecular oxygen in response to photoexcitation. As used herein, “photosensitizer” refers to a light-adsorbing molecule that when avtivated by light converts molecular oxygen into singlet oxygen. Suitable photosensitizers having lipophilic moieties are disclosed in the following references: Young et al, U.S. Pat. No. 6,375,930; and Young et al, U.S. patent application Ser. No. 2002/0006378, which are incorporated by reference. Additional photosensitizers that may be derivatized with lipophilic groups or capture moieties, such as biotin, and used with the invention are disclosed in the following references: Sessler et al, U.S. Pat. No. 5,292,414; Masuya et al, U.S. Pat. No. 5,344,928; McCapra, U.S. Pat. No. 5,705,622; Levy et al, 4,883,790; Meunier et al, U.S. Pat. No. 5,141,911; and the like, which are incorporated by reference. The following references disclose the use of conjugates between biotin and lipophilic moieties to anchor biotinylated molecules to membranes via an avidin or streptavidin: Plant et al, Anal. Biochem., 176:420-426 (1989); Bayer et al, Biochim. Biophys. Acta, 550: 464-473 (1979); Ramirez et al, J. Chromatogr. A, 971: 117-127 (2002); and the like, which are incorporated by reference.

A large variety of light sources are available to activate photosensitizers to generate singlet oxygen. Both polychromatic and monochromatic sources may be used as long as the source is sufficiently intense to produce enough singlet oxygen in a practical amount of time. The length of the irradiation is dependent on the nature of the photosensitizer, the nature of the cleavable linkage, the power of the source of irridiation, and its distance from the sample, and so forth. In general, the period for irridiation may be less than about a microsecond to as long as about 10 minutes, usually in the range of about one millisecond to about 60 seconds. The intensity and length of irradiation should be sufficient to excite at least about 0.1% of the photosensitizer molecules, usually at least about 30% of the photosensitizer molecules and preferably, substantially all of the photosensitizer molecules. Exemplary light sources include, by way of illustration and limitation, lasers such as e.g., helium-neon lasers, argaon lasers, YAG lasers, He/Cd lasers, and ruby lasers; photodiodes; mercury, sodium and xenon vapor lampls; incandescent lamps such as, e.g., tungsten and tungsten/halogen; flash lamps; and the like.

Examples of photosensitizers that may be utilized in the present invention are those that have the above properties and are enumerated in the following references: Turro, Modern Molecular Photochemistry (cited above); Singh and Ullman, U.S. Pat. No. 5,536,834; Li et al, U.S. Pat. No. 5,763,602; Ullman, et al., Proc. Natl. Acad. Sci. USA 91, 5426-5430 (1994); Strong et al, Ann. New York Acad. Sci., 745: 297-320 (1994); Martin et al, Methods Enzymol., 186: 635-645 (1990); Yarmush et al, Crit. Rev. Therapeutic drug Carrier Syst., 10: 197-252 (1993); Pease et al, U.S. Pat. No. 5,709,994; Ullman et al, U.S. Pat. No. 5,340,716; Ullman et al, U.S. Pat. No. 6,251,581; McCapra, U.S. Pat. No. 5,516,636; Wohrle, Chimia, 45: 307-310 (1991); Thetford, European patent publ. 0484027; Sessler et al, SPIE, 1426: 318-329 (1991); Madison et al, Brain Research, 522: 90-98 (1990); Polo et al, Inorganica Chimica Acta, 192: 1-3 (1992); Demas et al, J. Macromol. Sci., A25: 1189-1214 (1988); and the like. In one embodiment, photosensitizers used in the invention are porphyrins, e.g. as described in Roelant, U.S. Pat. No. 6,001,573, which is incorporated by reference. Many porphrins suitable for use with the invention are available commercially, e.g. Frontier Scientific, Inc. (Logan, Utah); Molecular Probes, Inc. (Eugene, Oregon); and the like.

### Separation of Released Molecular Tags

Molecular tags are electrophorectically separated to form an electropherogram in which the separated molecular tags are represented by distinct peaks. Methods for electrophoresis of are well known and there is abundant guidance for one of the ordinary skill in the art to make design choices for forming and separating particular pluralities of molecular tags. The follwing are exemplary references on electrophoresis: Krylov et al, Anal. Chem., 72: 111R -128R (2000); P. D. Grossman and J. C. Colburn, Capillary Electrophoresis: Theory and Practice, Academic Press, Inc., NY (1992); U.S. Pat. Nos. 5,374,527; 5,624,800; 5,552,028; ABI PRISM 377 DNA Sequencer User's Manual, Rev. A, January 1995, Chapter 2 (Applied Biosystems, Foster City, Calif.); and the like. In one aspect, molecular tags are separated by capillary electrophoresis. Design choices within the purview of those of ordinary skill include but are not limited to selection of instrumentation from several commercially available models, selection of operating conditions including separation media type and concentration, pH, desired separation time, temperature, voltage, capillary type and dimensions, detection mode, the number of molecular tags to be separated, and the like.

In one aspect of the invention, during or after eletrophoretic separation, the molecular tags are detected or identified by recording fluorescrnce signals and migration times (or migration distances) of the separated compounds, or by constructing a chart of relative fluorescent and order of migration of the molecular tags (e.g., as an electropherogram). To perform such detection, the molecular tags can be illuminated by standard means, e.g. a high intensity mercury vapor lamp, a laser, or the like. Typically, the molecular tags are illuminated by laser light generated by a He-Ne gas laser or a solid-state diode laser. The fluorescence signals can then be detected by a light-sensitive detector, e.g., a photomultiplier tube, a charged-coupled device, or the like. Exemplary electrophoresis detection systems are described elsewhere, e.g., U.S. Patent Nos. 5,543,026; 5,274,240; 4,879,012; 5,091,652; 6,142,162; or the like. In another aspect, molecular tags may be detected electrochemically detected, e.g. as described in U.S. Pat. No. 6,045,676.

Electrophoretic separation involves the migration and separation of molecules in an electric field based on differences in mobility. Various forms of electrophoretic separation include, by way of example and not limitation, free zone electrophoresis, gel electrophoresis, isoelectric focusing, isotachophoresis, capillary electrochromatography, and micellar electrokinetic chromatography. Capillary electrophoresis involves electroseparation preferably by electrokinetic flow, including electrophoretic, dielectrophoretic and/or electroosmotic flow, conducted in a tube or channel of from about 1 to about 200 micrometers, usually, from about 10 to 100 micrometers cross-sectional dimensions. The capillary may be long independent capillary tube or a channel in a wafer or film comprised of silicon, quartz, glass or plastic.

In capillary electroseparation, an aliquot of the reaction mixture containing the molecular tags is subjected to electroseparation by introducing the aliquot into an electroseparation channel that may be part of, or linked to, a capillary device in which the amplification and other reactions are performed. An electric potential is then applied to the electrically conductive medium contained within the channel to effectuate migration of the components within the combination. Generally, the electric potential applied is sufficient to achieve electroseparation of the desired components according to practices well known in the art. One skilled in the art will be capable of determining the suitable electric potentials for a given set of reagents used in the present invention and/or the nature of the cleaved labels, the nature of the reaction medium and so forth. The parameters for the eletroseparation including those for the medium and the electric potential are usually optimized to achieve maximum separation of the desired components. This may be achieved empirically and is well within the purview of the skilled artisan.

Detection may be by any of the known methods associated with the analysis of capillary electrophoresis columns including the methods shown in U.S. Pat. Nos. 5,560,811 (column 11, lines 19-30), 4,675,300, 4,274,240 and 5,324,401, the relevant disclosures of which are incorporated herein by reference. Those skilled in the electrophoresis arts will recognize a wide range of electric potentials or field strengths may be used, for example, fields of 10 to 1000 V/cm are used with about 200 to about 600 V/cm being more typical. The upper voltage limit for commercial systems is about 30 kV, with a capillary length of about 40 to about 60 cm, giving a maximum field of about 600 V/cm. For DNA, typically the capillary is coated to reduce electroosmotic flow, and the injection end of the capillary is maintained at a negative potential.

For ease of detection, the entire apparatus may be fabricated from a plastic material that is optically transparent, which generally allows light of wavelengths ranging from about 180 to about 1500 nm, usually about 220 to about 800 nm, more usually about 450 to about 700 nm, to have low transmission losses. Suitable materials include fused silica, plastics, quartz, glass, and so forth.

In one aspect of the invention, molecular tags are separated by electrophoresis in a microfluidics device, as illustrated diagrammnatically in FIGS. 9A-9C. Microfluldics devices are described in a number of domestic and foreign Letters Patent and published patent applications. See, for example, U.S. Pat. Nos. 5,750,015; 5,900,130; 6,007,690; and WO 98/45693; WO 99/19717 and WO 99/15876. Conveniently, an aliquot, generally not more than about 5 μl, is transferred to the sample reservoir of a microfluidics device, either directly through electrophoretic or pneumatic injection into an integrated system or by syringe, capillary or the like. The conditions under which the separation is performed are conventional and will vary with the nature of the products.

By way of illustration, FIGS. 9A-9C show a microchannel network 100 in a microfluidics device of the type detailed in the application noted above, for sample loading and electrophoretic separation of a sample of probes and tags produced in the assay above. Briefly, the network includes a main separation channel 102 terminating at upstream and downstream reservoirs 104, 106, respectively. The main channel is intersected at offset axial positions by a side channel 108 that terminates at a reservoir 110, and a side channel 112 that terminates at a reservoir 114. The offset between the two-side channels forms a sample-loading zone 116 within the main channel.

In operation, an assay mixture is placed in sample reservoir 110, illustrated in FIG. 9A. As noted, the assay mixture contains one or more target cells with surface-bound cleaving agent, one or more protein probes, and optionally,, molecular tag standard. The assay reaction, involving intial probe binding to target cell(s), followed by cleavage of probe linkers in probe-bound cells, may be carried out in sample resovoir 110, or alternatively, the assay reactions can be carried out in another reaction vessel, with the reacted sample components the added to the sample reservoir.

To load released molecular tags into the sample-loading zone, an electric field is applied across reservoirs 110, 114, in the direction indicated in FIG. 9B, wherein negatively charged released molecualr tags are drawn from reservoir 110 into loading zone 116, while uncharged or positively charged sample components remain in the sample reservoir. The released tags in the loading zone can now be separated by conbventional capillary electrophoresis, by applying an electric filed across reservoirs 104, 106, in the direction indicated in FIG. 9C.

### Peak Identification and Correlation

After electropherogram data is read by a processor, each peaks in the data is identified, or located by a single mirgration time. In the process of identifying peaks, conventional smoothing or filtering algorithms may be applied to remove noise and outlying data points that have no physical relevance, e.g. using moving average filters, Savitzky-Golay filters, or the like. Algorithms for such filters are disclosed in the following refernces: Numerical Recipes in C: The Art of Scientific Computing (Cambridge University Press, Cambridge, 1992); Hamming, Digital Filters, Second Edition (Prentice-Hall, Inc., Englewood Cliffs, N..J., 1983); and the like. Conventional peak identification algorithms may be employed to determine the locations and sizes of all peaks in the electropherogram data. A prefered peak identification algorithm is disclosed more fully below. As illustrated in FIG. 2D, the number of peaks identified may be larger than the number of molecular tags used in an assay. In the example of FIG. 2D, 22 peaks are identified, while only six molecular tags are used in the assay. Since the molecular tags and standards are predetermined beforehand emperically. Thus, for each molecular tag, an interval may be defined (referred to herein as a “migration interval”), as illustrated in FIG. 2D by the shaded rectangles below the electropherogram. The width of the migration interval may be defined in a variety of ways. For example, the center of each interval may correspond to an empirically determined mean value, referred to herein as the “emperical migration time” (shown as a vertical line in the shaded rectangles in the figure), and the width of the interval may be taken as twice the standard deviation, optionally multiplied by a user-defined value. Peaks whose locations fall outside of the migration intervals may be disregarded, as illustrated in FIG. 2E. In some intervals, e.g. (217) and (219), more than one peak location may be identified. the present invention provides a method for selecting among such peaks to make a correct correlation with a molecular tag.

In one embodiment of the invention, after all peaks are identified, as first electrophorectic standard is identified by determining the first peak that satisfied a set of necessary conditions based on known properties of the compound used as the standart, e.g. optical properties (it may be a different color than the molecular tags), quantity, known range of absolute migration times for the system used for electrophorectic separation, or the like. Preferably, a first electrophorectic standard is determined based on (i) the location of a peak within an empirically determined range, (ii) peak height exceeding a predetermined minimum value, and (iii) peak area exceeding a predetermined minimum value. In a preferred embodiment of the invention, a second electrophorectic standard is employed that has a longer migration time than any of the molecular tags employed in an assaym so that upon separation an electropherogram is produced similar to that illustrated in FIGS. 2A and 2B. Once the locations of both standards are determined, in one embodiment, migration times of molecular tags are determined as fractions of the interval defined by the two standards, as illustrated in FIG. 2B.

When multiple peaks have locations within the same migration interval, as illustrated in FIG. 2E, several methods may be employed to select a peak correlated with the molecular tag associated with the migration interval. In one embodiment, the location of each candidate peak is first determined relative to the first and second electrophorectic standards. For example, in as illustrated in FIG. 2F, two peaks are located at t₂₁ and t₂₂ within the migration interval centered at empirically determined, t₂. The following values are determined: S₁=(t₂₁-Tₛ₁)/(Tₛ₂-Tₛ₁) S₂=(t₂₂-Tₛ₁)/(Tₛ₂-Tₛ₁) The ratio, S₁ or S₂, that is closest to the ratio of the empirically determined migration time, T₂, and the difference between the migration times of the standards, that is T₂/(Tₛ₂-Tₛ₁), determines which candidate peak is correlated to the molecular tag of the migration interval.

In another embodiment, the location of each candidate peak is first determined relative to second electrophorectic standard and the previously determined peak location correlated with a molecular tag. For example, in as illustrated in FIG. 2F, two peaks are located at t21 and t22 within the migration interval centered at empirically determined, T₂. the following values are determined:

### S′₁=(t₂₁-Tₛ₁)/(Tₛ₂-Tₛ₁)

### S′₂=(t₂₂- T₁)/(Tₛ₂-T₁)

The ratio, S′₁, or S′₂, that is closest to the ratio of the empirically determined migration time, T₂, and the difference between the migration times of the second standard and T₁, that is, T₂/(Tₛ₂-T₁), determines which candidate peak is correlated to the molecular tag of the migration interval. In this embodiment, as peak locations are successively correlated to molecular tags, the most recent such identified migration time is used to select the next migration time when multiple peak locations are present in a migration interval. When no, or low levels of, molecular tag is generated in an assay, a corresponding peak may have a low signal-to-noise ratio and its location may be difficult to identify accurately. Therefore, for a peak location to be used as a standard, preferably such a peak has a signal-to-noise ratio above a minimal value. In one aspect, the minimum signal-to-noise ratio is at least 1.5, and preferably, at least 2.0, and more preferably, 2.5.

As mentioned above, peaks may be identified in electropherogram data in various ways, e.g. curve fitting, or the like. A preferred algorithm for determining peak location and other parameters, such as, peak height, peak size or area, and peak signal-to-noise ratio, is illustrated in FIGS. 2G to 2J and the flowchart of FIG. 3. As shown in FIG. 2G, a peak search window (210) is established having width (212). Window (210) scans (214) the entire data set by starting at the earliest (leftmost) time points, then after carrying out peak detection and analysis steps, the window (212) is shifted to the right a predetermined amount to an overlapping set of times for again carrying out the peak detection and analysis steps. This process continues until all of the data has been analyzed. The width of window (212), the amount shifted in each cycle of peak detection and analysis, are design choices within the ordinary skill in the art. After the position of peak search window (212) is established, a value for the local noise level, that is, the noise level within the search window, is determined as illustrated in FIGS. 2H and 2I. First, an average (222) is taken of all the data values, F(Xᵢ), in the window (220), after which all the data values in excess of the computed average are reduced to the average value (222), shown graphically (223) in FIG. 2I. This process is repeated and a new average value (226) is obtained. Again, data values (224) that exceed the new average (226) are reduced to the value of the new average. The process is repeated until there is effectively no change in the noise value, and the final noise value is taken as the local noise value (230) of the peak search window, as shown in FIG. 2J. Once this value is obtained, the peak location is taken as the ordinate, or migration time value, Xₘₐₓ, that corresponds to the maximum data value, F(Xⱼ), in the peak search window; the peak starting location, tₛₜₐᵣₜ, (236) is the ordinate corresponding to the intersection (232) of the noise level (230) and F(X); the peak ending location, t_{end}, (240) is the ordinate corresponding to the intersection (234) of the noise level (230) and F(X); peak width is the difference between the peak ending and the peak start; and the peak signal-to-noise ratio is the ratio of the peak height, F(Xₘₐₓ), to the noise value (230). Optionally, after the peak location is determined, the noise value may be re-computed (308, FIG. 3) with the peak search window re-centered at Xₘₐₓ. After a peak location is determined, refinements in the baseline value of the local noise may be made. For example, local noise values may be computed adjacent to peak start and peak end points to determine the slope of a baseline of the peak. Such a value may then be used in computing a more accurate value of peak area. After such peak parameters are computed, certain necessary conditions (314) must be met before peak area is determined and the next window shift implemented. Necessary conditions include that the peak width does not overlap other peak widths, that the peak width is wider than a pre-set minimum, e.g. no process were implemendted to remove spurious spikes and other outlying values from the electropherogram data. Preferably, peak area is determined by calculating the time-normalized area, that is, the value: PA=E[F(Xᵢ)/Xᵢ]for i=tₛₜₐᵣₜ, t_{end}

### Dynamic Range Extension

As illustrated in FIG. 4A, in certain situations, an instrument will detect and record electropherogram data in multiple channels, e.g. fluorescence intensity within different wavelength ranges, and at the same time signal intensity values will be outside the dynamic range (403) of the detectore in one or more of the channnels. When this occurs, information about signal intensity is lost in regions of electropherogram data from a particular channel (404 and 406 in FIG. 4). However, if, in another channel, the signal does not exceed the dynamic range of the detector, then the “unsaturated” signal of the other channel may be used to estimate the signal values in the “saturated” regions of the first channel. Such a method is shown diagrammatically in FIG. 4A and by a flowchart in FIG. 4B. First, the unsaturated peak that corresponds to a “saturated” peak (404) is determined (408 and 410 in FIG. 4A, 432 in FIG. 4B), after which data values immediately adjacent to the regions of saturation (414) and (412) are mapped (416) onto the corresponding regions of the unsaturated peak by a linear function (434). Other functions could be used if a detector responded nonlinearly to increases in signal intensity. Once such a functional relationshhip is established, then the data values of the first channel in the “saturated” region are estimated by extrapolation,

### Computer System and Programs

A computer preferably performs steps of the method of identifying peaks in electropherogram data described above. In one embodiment, a computer comprises a processing unit, memory, I/O device, and associated address/data bus structures for communicating information therebetween. The processing unit may be a conventional microprocessor driven by an appropriate operating system, including RISC and CISC processors, a dedicated microprocessor using embedded firmware, or a customized digital signal processing circuit (DSP), which is dedicated to the specific processing tasks of the method. The memory may be within the microprocessor, i.e. level 1 cashe, fast S-RAM, i.e. level 2 cashe, D-RAM, or disk, either optical or magnetic. The I/O device may be any device capable of transmitting information between the computer and the user, e.g. a keyboard, mouse, network card, or the like. The address/data bus may be a PCI bus, NU bus, ISA, or any other like bus structure. When the computer performs the method of the invention, the above-described method steps are embodied in a program stored in or on a computer-readable product. Such computer-readable product may also include programs for graphical user interfaces and programs to change settings on electrophoresis systems or data collection devices.

### EXAMPLE 1

### Algorithm for Correlating peaks to Molecular Tags

### Using Previously Correlated Peaks as Standards

Purpose: to identify molecular tags in two-marker strategy by using newly identified molecular tag as standard dynamically.

Formula: The new algorith needs to use a funtion, called NRD(e₁, p₁, e₂, p₂)·e₁ and e₂ are molecular tage p₁ and p₂ are peaks in a trace. It calculates the normalized difference of ratio p₂ to p₁ and ratio e₂ to e₁ as following:

NRD(e₁, p₁, e₂, p₂)=Abs(((tₚ₂-t₁)/(tₚ₁-t₁)-mₑ₂/mₑ₁)/(mₑ₂-mₑ₁)),

Where tₚ₁, tₚ₂ are absolute migration time of peak p₁ and p₂ respectively, t₁ is the absolute migration time of marker M1, mₑ₁, mₑ₂ are mean relative migration time of molecular tag e₁ and e₂ respectively in database. In addition, (tₚ₂-t₁)/(tₚ₁-t₁) equals the relative migration time ration of p₂ to p₁. (This normalization assumes the SD is linear to mₑ₂-mₑ₁)

Step-By-Step Algorithm:

1. Let PreTag=empty, PrePeak=empty, PreTag2=empty, PrePeak2=empty, FastestPeak =M1 peak

2. For each molecular tag ei in the target list (from fastest to slowest):

2.1. SlowestPeak=M2 peak

2.2. If molecular tag eᵢ is already manually assigned to a peak pₐ,:

2.2.1. PreTag=eᵢ, PrePeak=Pₐ

2.2.2. PreTag2=empty, PrePeak2=empty

2.2.3. FastestPeak=Pₐ

2.2.4. Go to step 2

2.3. If there are molecular tags slower than eᵢ; and are already manually assigned to peaks:

2.3.1. Find the manually assigned molecular tag eₐ, which is closest to eiand is slower than eᵢ, and let SlowestPeak=eₐ,

2.4. Find all the peaks in the migration interval, i.e. tₚⱼ-mₑᵢσₑᵢ<θand tₚⱼ is between FastestPeak and SlowestPeak, and the peak is not manually unassigned to molecular tag eᵢ (this is not applicable due to the current definition of manual assignment), and compose a candidate peak set, CP={c₁, c₂, . . . , Cₖ), of molecular tag eᵢ

2.5. If CP is empty, do not assign eᵢ to any peak, and go to step 2

2.6. If PreTag =empty (no molecular tag is assigned to a peak yet):

2.6.1. Find the peak cⱼ in CP with smallest t_{cj}-mₑᵢσₑᵢ and assign eᵢ to the peak cⱼ

2.6.2. PreTag=eᵢ, PrePeak=cⱼ

2.7. If PreTag is not empty (use PreTag as standard to identify eit:

2.7.1. Calculate the normalized ratio difference of eᵢ for each cⱼ to the previously identified molecular tag, PreTag, by function NRD(PreTag, PrePeak, eᵢ, cⱼ)

2.7.2. If PreTag2 is not empty (use the PreTag2 to double check that the previously assigned molecular tag is a right assignment):

2.7.2.1. Calculate NRD(PreTag2, PrePeak2, eᵢ, cⱼ) for each cⱼ in CP

2.7.2.2. Find cₖ with smallest NRD(PreTag2, PrePeak2, cₖ)

2.7.2.3. If the peak cₖ is PrePeak (which means the peak cₖ is already assigned to be PreTag) and NRD(PreTag2, PrePeak2, eᵢ, cₖ)<NRD(PreTag2, PrePeak2, PreTag, cₖ) (the pre-assigned peak is a better fitting to molecular tag e,):

2.7.2.3.1. Assign molecular tag ei to peak cₖ (then, cₖ is not assigned to PreTag anymore)

2.7.2.3.2. PreTag=eᵢ, PrePeak=cₖ(PreTag2 and PrePeak2 do not change)

2.7.2.3.3. go to step 2

2.7.2.4. Else if cₖ migrates faster than PrePeak and NRD(PreTag2, PrePeak2, eᵢ, cₖ) <NRD(PreTag2, PrePeak2, PreTag, PrePeak) (there is a peak faster than pre-assigned peak and it should be assigned to eⱼ with more confidence, so the pre-assigned peak is un-assigned since PreTag migrates faster than eᵢ):

2.7.2.4.1. Assign eᵢ to cₖ

2.7.2.4.2. Un-assign PreTag to Prepeak

2.7.2.4.3. PreTag=eᵢ, PrePeak=cₖ

2.7.2.4.4. go to step 2

2.7.2.5. Else (the peak cₖ doesn't conflict with PreTag and PrePeak):

2.7.2.5.1. Find the peak cₗ (which could be cₖ or not) with the smallest NRD(PreTag, PrePeak, eᵢ c) and cₗ is slower than PrePeak in CP

2.7.2.5.3. Assign molecular tag ei to peak cₗ

2.7.2.5.4. PreTag2=PreTag, PrePeak2=PrePeak

2.7.2.5.5. PreTag=eᵢ, PrePeak=cₗ

2.7.2.5.6. FastestPeak=PrePeak2

2.7.2.5.7. go to step 2

2.7.3. Else (PreTag2 is empty),lk

2.7.3.1. Find the best peak cⱼ in CP, which has the smallest t_{cj}-mₑᵢ/σₑᵢ(to double check the previously assigned molecular tag is a right assignment)

2.7.3.2. If cj is PrePeak or migrates faster than PrePeak and t_{cj}-mₑᵢ/σₑᵢ<t_{PreTag}-M_{PreTag}/σ_{PreTag}(there is a conflicting peak fitting ei better, and the previous assigned molecular tag should be called off)

2.7.3.2.1. Assign molecular tag eᵢ to peak cⱼ

2.7.3.2.2. Un-assign PreTag to PrePeak

2.7.3.2.3. PreTag =ei, PrePeak =cⱼ

2.7.3.2.4. Go to step 2

2.7.3.3. Else, do not change the previous assignment

2.7.3.4. Find the peak cₗ with the smallest NRD(PreTag, PrePeak, eᵢ, cₗ) and cₗ is slower than PrePeak in CP

2.7.3.5. If no cl is selected, do not assign the molecular tag ei to any peak, and go to step 2

2.7.3.6. Assign molecular tag ei to peak cl

2.7.3.7. PreTag2 = PreTag, PrePeak2 = PrePeak

2.7.3.8. PreTag = ei, PrePeak = cl

2.7.3.9. FastestPeak = PrePeak2

2.7.3.10. go to step 2

1. It is assumed that all the molecular tags are between M1 and M2. And the relative migration time is calculated as (t-t1)/(t2-t1), where t, t1, t2 is the migration time of a peak, M1 and M2 respectively and M1 is the faster marker.

2. This method helps to prevent the following misidentification a. An expected molecular tag is assigned to a wrong peak instead of the right peak when both the peaks are in the migration interval of the molecular tag b. An expected molecular tag is not assigned to the right peak (when the peak is in the migration interval of the molecular tag), since the right peak is incorrectly assigned another molecular tag due to a.

3. This method doesn't prevent a junk peak to be assinged an un-expected molecular tag.

4. A molecular tag ei is assigned to peak pj if the following condition is satified: a. Peak pj is in molecular tag ei migration interval, i.e. tpj-mei/σei <θAND one of the following conditions is satified a. ei is the best fit to pj and pj is the best fit to ei b. ei is the best fit to pj and pj is the not best fit to ei, but the best fit to ei is a better fit to another molecular tag and is assigned to the other molecular tag c. pj is the best fit to ei and ei is not the best fit to pj, but the best fit to pj is a better fit to another peak and is assigned to the other peak

### EXAMPLE 2

### Dynamic Range Extension Algorithm

Purpose: Extrapolate signal values of electropherogram data in “saturated” regions. Inputs: (i) A one-dimensional array P containing the signal intensity of saturated peak, where po is the peak starting, and pn-l is the peak ending, n is the width of the saturated peak and thus the size of P. (ii) A one-dimensional array D containing the signal intensity of corresponding peak in dynamic channel. (iii) S: saturated peak threshold

Step-By-Step Algorithm:

1. Find the boundary of saturated range in array P. a. Find the first data point pi in P such that pi <S and pi>S, and denote the index of this data point as k b. Find the last data point pj in P such that pj <S and pj-l >S, and denote the index of this data point as l

2. compose array Y (pk-6, pk-5, ...,pk-1,p1‘,P1’, ...,p1≢), and array X = (dk-6, dk-5, ...,dk-1, d1‘, d1’, ...,d1≢). If k-6 <0, the starting from 0; if 1≢>n-1, then ending at n-1 3. Find the best linear fitting y = A*x B for Y and X as following: a. B = (Σ(xi*yi)-m*xavg*yavg)/(Σ(xi*xi)-m*xavg*xavg), where xavg and yavg are the average of X and Y respectively, m is the size of X b. A = yavg-B*xavg 4. Calculate the recovered peak array P. For j = k to l, pj =A*dj B.

### EXAMPLE 3

### Identification of Multiple Cytokines

In this example, seven human sytokines, IL-1a, IL-2, IL-4, IL-6, IL-8, TNFa, and IFNy, were detected in a single multiplexed sandwich assay as illustrated in FIG. 1A, with the exception that only a single molecular tag was conjugated to each antibody. 10 μL of cytokine sample containing 333 pM of each cytokine is combined with 20 μL of binding composition comprising a mixture of antibodies specific for each of the cytokines. wherein each antibody is conjugated to a different molecular tag (identified in FIG. 11) by a singlet oxygen-cleavable linkage. After incubation for 30 min, 10 μl of cleaving agent (a biotinylated seconds anti-cytokine antibody that after incubation is combined with avidinated photosensitizer beads (Perkin Elmer Life Sciences, Boston, MA, also disclosed in International patent publication WO 01/90399) is added and the mixture is incubated for an additional 30 min. The reaction buffer is exchanged twice with 100 μL of low salt buffer by drawing fluids through a filter under vacuum, after which 50 μL of electrophorectic separation medium containing two electrophorectic standards is added and the mixture is irradiated for 5 min (e.g. using a high power GaAIAs IR emitter such as model number OD-880W manufactured by OPTO DIODE Corp, Newbury Park, Calif.). After 5 minute of shaking, 10 μL of the mixture contatining released molecular tags is transferred to an Applied Biosystems (Foster City, Calif.) model 3100 capillary electrophoresis systems for generating the electropherogram data displayed in FIG. 11.

Peaks in the electropherogram data are identified and correlated with the indicated molecular tags by programming in a convenient language, such as C♯ or VB.NET, or the like, a conventional Pentium-based computer to carry out the algorithm of Example 1.

What is claimed is:

1. A computer-readable product embodying a program for execution by a computer to identify a plurality of molecular tags by determining locations of peaks in electropherogram data correlated with such tags, the program comprising instructions for:
(a) reading electropherogram data from a storage medium, the electropherogram data obtained by electrophoretic separation of the plurality of molecular tags and one or more electrophoretic standards, each electrophoretic standard and molecular tag having a different electrophoretic mobility such that upon electrophoretic separation each electrophoretic standard and molecular tag forms a distinct peak in the electropherogram data, and each peak of such one or more electrophoretic standards and molecular tags having a migration interval and each migration interval having a mean;
(b) determining a peak location of at least one electrophorctic standard within a migration interval in the electropherogram data;
(c) determining peak locations of peaks within a migration interval in the electropherogram data closest to an electrophoretic standard or a qualified peak in a qualified peak set, the peak locations being relative to the location of the closest electrophoretic standard or qualified peak, and correlating with a molecular tag or an electrophoretic standard a peak within the migration interval whose location is closest to the mean of the migration interval;
(d) determining a peak signal-to-noise ratio of the peak and adding the peak to the qualified peak set if the peak signal-to-noise ratio is greater than or equal to 1.5; and
(e) repeating steps (c) and (d) until a peak location is correlated to every molecular tag having a peak in the electropherogram data.

2. The computer-readable product of claim I wherein said step of reading includes prvding at least two said electrophoretic standards, a first electrophoretic standard having an electrophoretic mobility higher than any of said electrophoretic mobilities of said molecular tags and a second electrophoretic standard having an electrophoretic mobility lower than any of said electrophoretic mobilities of said molecular tags.

3. The computer-readable product of claim 2 wherein each of said locations of said peaks correlated with said molecular tags is determined as a ratio of the difference between a migration time of such peak and a migration time of said closest electrophoretic standard or qualified peak and the difference between a migration time of said second electrophoretic standard and the migration time of said closest electrophoretic standard or qualified peak.

4. The computer-readable product of claim 1 further including a step of determining for each said molecular tag a peak size and correlating the peak size to an amount of said molecular tag.

5. A computer system for identifying a plurality of molecular tags by determining locations of peaks in electropherogram data correlated with such molecular tags, the computer system comprising:
an input device for inputting electropherogram data from a storage medium, the electropherogram data obtained by electrophoretic separation of the plurality of molecular tags and one or more electrophoretic standards, each electrophoretic standard and molecular tag having a different electrophoretic mobility such that upon electrophoretic separation each electrophoretic standard and molecular tag forming a distinct peak in the electropherogram data, and each peak of such one or more electrophoretic standards and molecular tags having a migration interval and each migration interval having a mean;
a memory for storing the electropherogram data; and
a processing unit programmed for:
(a) determining a peak location of at least one electrophoretic standard within a migration interval in the electropherogram data;
(b) determining peak locations of peaks within a migration interval in the electropherogram data closest to an electrophoretic standard or a qualified peak in a qualified peak set, the peak locations being relative to the location of the closest electrophoretic standard or qualified peak, and correlating with a molecular tag or an electrophoretic standard a peak within the migration interval whose location is closest to the mean of the migration interval;
(c) determining a peak signal-to-noise ratio of the peak and adding the peak to the qualified peak set if the peak signal-to-noise ratio is greater than or equal to 1.5;
(d) repeating steps (b) and (c) until a peak location is correlated to every molecular tag having a peak in the electropherogram data.
